(19) United States
(12) Patent Application Publication
(10) Pub. No.: US 2022/0056002 A1
(43) Pub. Date: Feb. 24, 2022

(54) PHOTOCLEAVABLE LINKER FOR CATCHING AND/OR RELEASING OF CIRCULATING TUMOR CELLS OR EXTRA CELLULAR VESICLES

(71) Applicant: The University of Kansas, Lawrence, KS (US)

(72) Inventors: Steven Allan Soper, Baton Rouge, LA (US); Blake R. Peterson, Columbus, OH (US); Richard S. Givens, Lawrence, KS (US)

(73) Assignee: The University of Kansas, Lawrence, KS (US)

(21) Appl. No.: 17/416,172

(22) PCT Filed: Dec. 20, 2019
(86) PCT No.: PCT/US2019/068127

§ 371 Date: Jun. 18, 2021

Related U.S. Application Data
(60) Provisional application No. 62/784,003, filed on Dec. 21, 2018.

(51) Int. Cl. C07D 311/16; G01N 33/547

(52) U.S. Cl. CPC C07D 311/16; G01N 33/547

(57) ABSTRACT

A photocleavable heterobifunctional linker can include a structure of Formula (A) wherein coumarin is any coumarin or coumarin derivative; R, R⁹, and R¹⁰ are each independently a chemical moiety; R¹ is a hydrogen, protecting group, leaving group, substrate, or capture entity; R² is a hydrogen, hydroxyl, halide, alkoxy, anhydride, amino, protecting group, leaving group, substrate, or capture entity; L¹ is a sub-linker; and L² is a sub-linker. A capture device can include the photocleavable bifunctional linker having a structure of Formula (A) as provide herein, wherein R¹ is a substrate. A method of capturing a target substance can include: providing the capture device having the photocleavable bifunctional linker with the structure of Formula (A) and contacting a target substance to the capture moiety such that the target substance is captured. Irradiating the linker with light can cleave the linker, thereby releasing the target substance from the substrate.

### CROSS-REFERENCE

This patent application claims priority to U.S. Provisional Application No. 62/784,003 filed Dec. 21, 2018, which provisional is incorporated herein by specific reference in its entirety.

### GOVERNMENT RIGHTS

This invention was made with government support under grant number EB020594 awarded by the National Institutes of Health. The government has certain rights in the invention.

### BACKGROUND

### Field

The present disclosure relates to photocleavable linkers that include chemical structures that are cleaved when exposed to certain wavelengths of light. More particularly, the present disclosure relates to photocleavable linkers that are derived synthetic amino acids that include a coumarin group between an amine group and a carboxylic acid group.

### Description of Related Art

Previously, linkers have been used to link different chemical structures together or otherwise attach a first substance (e.g., chemical, protein, peptide, etc.) to a second substance (e.g., different from first substance). Linkers are available with a wide variety of structures for many different purposes. In some instance, linkers are designed to be a permanent link between different chemical structures, where the linker is resistant to being cleaved or separated from one of the chemical structures. In other instances, linkers are designed to be cleavable upon action by a stimulus, which degrades the linker in some chemical reaction to allow for separation of the two different chemical structures from each other. In biological applications and many assays, it can be preferable to have a linker that is stable under certain conditions, but which can be cleaved under other conditions, such as exposure to a specific stimulus. Cleavable linkers have found a variety of uses in the biological and diagnostic arts.

Many cleavable linker materials have been developed for use in affinity-selection assays to allow for linking of two different substances, such as by one substance capturing another substance. These types of cleavable linkers also allow for selective dissociation of the two different chemical substance, such allowing the release a captured element. An exemplary linker employed a single-stranded DNA oligonucleotide linker that contained: (i) a 3′-primary amine for immobilization to carboxylic acid (COOH) groups on solid surfaces via EDC/NHS coupling reagents; (ii) a 5′-disulfide group that when reduced to a sulfhydryl would covalently tether maleimide-labeled antibodies (Abs); and (iii) an internal dU residue within the single-stranded DNA that could be cleaved after affinity-selection via the USER™ (uracil specific excision reagent) enzyme system to release affinity-purified biomarkers. The enzymatic release strategy was first employed in a microfluidic device for the catch and release of circulating tumor cells (CTCs). There was a demonstrated efficient recovery (85%) of SKBR3 by anti-EpCAM selection and high release efficiency (90%) of affinity-selected cells without biological damage (>85% cell viability).

While the enzymatic system is efficient and does not damage rare biomarkers, there are issues for integrating this release strategy in different linking protocols. The enzyme requires strict temperature regulation to preserve activity (4° C.) before use, and excessive enzyme use must be minimized due to the high cost of the enzyme; both factors incur significant engineering complexity when using this enzymatic approach. Further, in applications where processing time is critical (e.g., stroke diagnosis), the enzyme's incubation time (˜30 min) for biomarker release must be reduced.

In view of the foregoing, there is a need for an improved system for capturing and releasing chemical and biological substances.

### SUMMARY

In some embodiments, a photocleavable heterobifunctional linker can include a structure of Formula A,

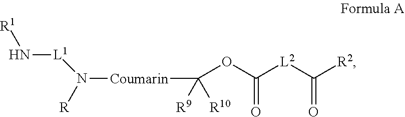

wherein: coumarin is any coumarin or coumarin derivative; R, R⁹, and R¹⁰ are each independently a chemical moiety; R¹ is a hydrogen, protecting group, leaving group, substrate, or capture entity; R² is a hydrogen, hydroxyl, halide, alkoxy, anhydride, amino, protecting group, leaving group, substrate, or capture entity; L¹ is a sub-linker; and L² is a sub-linker.

In some embodiments, a method of synthesizing a photocleavable heterobifunctional linker can include: providing a coumarin having the following structure,

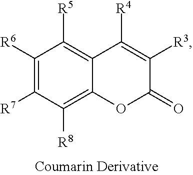

wherein one of R³-R⁸ is a leaving group and another of R³-R⁸ is a protecting group and the rest of R³-R⁸ are each individually a chemical moiety; reacting the leaving group with a precursor of a first linker arm such that the first linker arm replaces the leaving group,

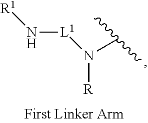

wherein R is a chemical moiety and R¹ is an amine protecting group; converting the protecting group to an alcohol group; and reacting the alcohol group with a precursor of a second linker arm so as to form an ester with the oxygen of the alcohol group in the second linker arm,

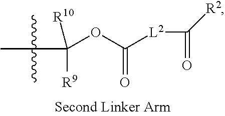

wherein R² is a carbonyl protecting group, R9 and R10 are as defined for R, and exemplified by hydrogen, alkyl, cycloalkyl, aryl or other chemical moiety.

In some embodiments, a capture device can include the photocleavable bifunctional linker having a structure of Formula A as provide herein, wherein: coumarin is any coumarin or coumarin derivative; R, R⁹, and R¹⁰ are each independently a chemical moiety; R¹ is a substrate; R² is a hydrogen, hydroxyl, halide, alkoxy, anhydride, amino, protecting group, leaving group, or capture entity; L¹ is a sub-linker; and L² is a sub-linker.

In some embodiments, a method of capturing a target substance can include: providing the capture device having the photocleavable bifunctional linker with the structure of Formula A, wherein the R² is the capture entity; and contacting a target substance to the capture moiety such that the target substance is captured.

In some embodiments, a method of releasing a captured target substance can include: providing the capture device having the photocleavable bifunctional linker with the structure of Formula A, wherein the R² is the capture entity having a target substance associated therewith; and irradiating the photocleavable heterobifunctional linker with light that cleaves the linker, thereby releasing the target substance from the substrate.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A shows reaction Scheme 1 for synthesizing a photocleavable bifunctional linker.

FIG. 1B shows a scheme for linking the a photocleavable bifunctional linker to a substrate, then linking the linker to a capture entity, capturing a target substance with the capture entity, and releasing the target substance by photocleaving the a photocleavable bifunctional linker.

FIG. 1C shows a scheme for photocleaving the a photocleavable bifunctional linker.

FIG. 1D shows ultra-high performance liquid chromatography (UPLC) of the photocleavage of the linker using 400-450 nm light for exposure times of 0, 1, 2, and 10 min.

FIG. 1E shows the UV/vis spectra of the intact photocleavable linker as a function of exposure time.

FIG. 1F shows the fluorescence emission spectra of the photo-irradiated linker as a function of time.

FIG. 2A shows a scheme for linking Cy5 to the UV/O₃-activated COC substrate.

FIG. 2B shows data for modified and UV/O₃-activated COC substrates in different solvents for water contact angle, carboxylic acid density, and ATR-FTIR peak area for C═O and OH groups.

FIG. 2C shows ACN pre-treatment of UV/O3-COC yielded higher loads of Cy5-oligonucleotides compared to MES (N=33-35).

FIG. 2D shows immobilization in solvent provides further gains that were observed in fluorescence microscopy by EDC/NHS coupling in ACN (N=4).

FIG. 3A shows the LED's spectral output, the absorbance spectra of the PC linker (measured at 526 μM in PBS, pH 7.4), and the Rubylith® film used to protect devices from ambient light and premature photocleavage.

FIG. 3B shows that the results of the LED's spatial flux by being measured by rastering a sensor underneath the LED and fitting a 2-dimensional Gaussian (R2=0.9986), which showed a uniform exposure (34±4 mW/cm2) over the device.

FIG. 3C shows data for when the linker was immobilized at three concentrations (reaction excesses of 5×, 1×, and 0.2×) and labeled with Cy5-oligonucleotide fluorescent reporter by EDC/NHS conjugation, then the device was exposed beneath the LED for 10 min to cleave the linker and release the Cy5-oligonucleotide.

FIG. 3D shows the amount of Cy5-oligonucleotide released after different exposure times was collected in the effluent quantified by fluorometry.

FIG. 4A shows data for the captured cell numbers per device for the direct Ab coupled or photocleavable heterobifunctional linker (Ab-PC), and after release of the Ab-PC.

FIG. 4B includes images that indicate the intact nature of the cells following processing use the CTC assay.

FIG. 4C shows data for the release efficiency for both CTC types was >90% using a 2 min blue light exposure.

FIG. 4D shows data for viability of the CTC following capture and release.

FIG. 5A shows the performance of sinusoidal microfluidic device using PC linker for anti-EpCAM enrichment of SKBR3 cells spiked into whole blood (N=3).

FIG. 5B shows that LED release had no effect on viability.

FIGS. 5C-5E show the released cells in culture for 2-96 h (Scale bars=100 μm).

FIG. 5F shows the relative 8-oxo-G damage for DNA and RNA.

FIG. 5G shows that no treatment altered the gene mRNA expression compared to controls.

FIG. 6A shows the number of nanoparticles released by LED exposure to the photocleavable linker by NTA.

FIG. 6B shows the number of nanoparticles released by LED exposure to the photocleavable linker by TEM imaging.

FIG. 6C shows ddPCR analysis of 5 genes that are known to be dysregulated as a result of ischemic stroke.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part thereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology includes photocleavable chemical structures that can be used as heterobifunctional linkers for linking a first substance to a second substance. The photocleavable chemical structure can be configured for being stable under certain conditions and then cleave into two portions when exposed to a select stimulus light to allow separation of the first substance from the second substance. This selective cleavage allows for selective linking of the two substances and then the selective dissociation of the two substances from each other. This property can be beneficial in chemical linking or capturing of substances. For example, the first substance can be a chemical entity (e.g., liposome) that is desired to be linked to a different chemical entity (e.g., receptor-targeting ligand) to allow for the targeting ligand to bind with the targeted receptor such that the liposome is attached through the linker to the receptor, whether or not the receptor is associated with a cell or free. In another example, the first substance can be a substrate (e.g., well bottom, particle, magnetic bead, or the like) and the second substance can be a biomolecule that functions as a capture entity (e.g., antibody, protein, nucleic acid, aptamer, or other). Accordingly, it should be recognized that at least one of the substances can be an anchor substance and the other substance a free substance that moves around in a medium having the anchor substance. Also, it should be recognized that both substances may be anchor substances, or both substances may be free substances that move around in a medium together. The purpose of the cleavable linker allows for selective linking of two elements and then selective cleaving for dissociation of the two elements from each other.

The photocleavable heterobifunctional linker can be used in various biologically related platforms and assays. The photocleavable heterobifunctional linker can be used in microfluidic affinity enrichment, such as by having one end linked to a substrate and the other end linked to a capture entity, such as antibody, protein, nucleic acid, aptamer, or other. The capture agent can be selected so that it has affinity for and captures a target substance, such as a biomarker. Accordingly, the capture entity can be tailored depending on the desired target to be captured. Examples of biomarkers can include circulating cells (e.g., circulating tumor cells for epithelial cancers or CD8(+) T-cells for stroke), extracellular vesicles, proteins, nucleic acids, or others. The photocleavable functionality allows for selective release of the target biomarker, which can be analyzed to obtain clinically relevant information for a disease state.

In some embodiments, the heterobifunctional linker can be a photocleavable linker with two different functional groups. Accordingly, the photocleavable linker can include a first functional group and an opposite second functional group that is different from the first functional group. The photocleavable linker also includes a photocleavable moiety between the first functional group and second functional group. The photocleavable moiety can include at least one bond that is photocleavable with light having a wavelength within a range of wavelengths that are stimuli for the photocleavable moiety. For example, the light can have a wavelength that is greater than about 380 nm. In some aspects, the linker is elongate with the photocleavable moiety between the first functional group and the second functional group. The first functional group can be coupled to a first end or first side of the photocleavable moiety and the second functional group can be coupled to a second end or second side of the photocleavable moiety.

In some embodiments, the photocleavable moiety includes a chromophore group, such as a coumarin group. In some aspects, the coumarin group can have a photocleavable ester. In some aspects, the coumarin group can have a photostable amine and a photocleavable ester. In some aspects, the coumarin group can have a photostable amine on the first side or first end of the coumarin group and a photocleavable ester on the second side or second end of the coumarin group.

In some embodiments, the first functional group includes an amine group that is reactive. The amine group can be a primary amine group or secondary amine group, which includes at least one hydrogen bound to the nitrogen for reactive potential. Notably, the hydrogen is removed when the nitrogen of the first functional group is linked to a first substance. The secondary amine can include a non-reactive R group, such as a chemical moiety as defined herein.

In some embodiments, the second functional group includes a carbonyl group that is reactive and includes a leaving group. For example, the carbonyl group can be an aldehyde (e.g., hydrogen leaving group), carboxylic acid (e.g., hydroxyl leaving group), acid halide (e.g., halide leaving group), ester (e.g., alkoxy leaving group), or acid anhydride (e.g., carbonyl leaving group), as well as possibly an amide (e.g., amine leaving group) in some instances.

In some embodiments, the heterobifunctional linker can include a structure of Formula A or derivative thereof:

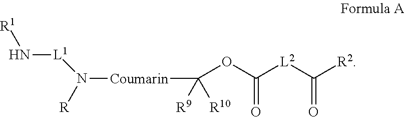

In Formula A, the R, R⁹, and R¹⁰ can each independently be any chemical moiety from hydrogen to other chemical substituents, the R¹ can be a hydrogen or an amine protecting group or leaving group, and the R² can be a carbonyl protecting group or leaving group.

The L¹ and L² in Formula A represent linkers, which are sub-linkers of the heterobifunctional linker. These linkers can be various chemical structures that separate the coumarin from the amine functional group (first functional group) and the carbonyl functional group (second functional group), wither without leaving groups or protecting groups.

In some embodiments, the heterobifunctional linker can include a structure of Formula A1 or derivative thereof:

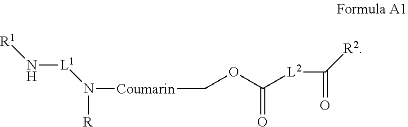

In Formula A1, the R can be any chemical moiety from hydrogen to other chemical substituents, the R¹ can be a hydrogen or an amine protecting group or leaving group, and the R² can be a carbonyl protecting group or leaving group.

The L¹ and L² in Formula A1 represent linkers, which are sub-linkers of the heterobifunctional linker. These linkers can be various chemical structures that separate the coumarin from the amine functional group (first functional group) and the carbonyl functional group (second functional group), wither without leaving groups or protecting groups.

In some embodiments, the R, R⁹, and R¹⁰ groups can independently of each other include any possible substituent or one substituent or a combination of the substituents recited herein. In some aspects, the R, R⁹, and R¹⁰ groups can independently of each other include hydrogen, halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, derivatives thereof, any substituted or unsubstituted, or combinations thereof as well as other well-known chemical substituents.

In some embodiments, the R, R⁹, and R¹⁰ groups can independently of each other include hydrogen, alkyl, alkenyl, alkynyl, aryl, alkaryl, aralkyl, halo, hydroxyl, sulfhydryl, alkoxy, alkenyloxy, alkynyloxy, aryloxy, acyl, alkylcarbonyl, arylcarbonyl, acyloxy, alkoxycarbonyl, aryloxycarbonyl, halocarbonyl, alkylcarbonato, arylcarbonato, carboxy, carboxylato, carbamoyl, mono-(alkyl)-substituted carbamoyl, di-(alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(alkyl)-substituted amino, mono- and di-(aryl)-substituted amino, alkylamido, arylamido, imino, alkylimino, arylimino, nitro, nitroso, sulfo, sulfonato, alkylsulfanyl, arylsulfanyl, alkylsulfinyl, arylsulfinyl, alkylsulfonyl, arylsulfonyl, phosphono, phosphonato, phosphinato, phospho, phosphino, any with or without hetero atoms, any including straight chains, any including branches, and any including rings, derivatives thereof, any substituted or unsubstituted, and combinations thereof.

In some embodiments, the R, R⁹, and R¹⁰ groups can independently of each other include hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO) O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-substituted arylcarbamoyl (—(CO)—NH-aryl), di-substituted arylcarbamoyl (—(CO)—NH-aryl)₂, thiocarbamoyl (—(CS)—NH₂), mono-(C₁-C₂₄ alkyl)-substituted thiocarbamoyl (—(CS)—NH(C₁-C₂₄ alkyl)), di-(C₁-C₂₄ alkyl)-substituted thiocarbamoyl (—(CS)—N(C₁-C₂₄ alkyl)₂), mono-substituted arylthiocarbamoyl (—(CS)—NH-aryl), di-substituted arylthiocarbamoyl (—(CS)—NH-aryl)₂, carbamido (—NH—(CO)—NH₂),), mono-(C₁-C₂₄ alkyl)-substituted carbamido (—NH(CO)—NH(C₁-C₂₄ alkyl)), di-(C₁-C₂₄ alkyl)-substituted carbamido (—NH(CO)—N(C₁-C₂₄ alkyl)₂), mono-substituted aryl carbamido (—NH(CO)—NH-aryl), di-substituted aryl carbamido (—NH(CO)—N-(aryl)₂) cyano(—C≡N), isocyano (—N⁺≡C⁻), cyanato (—O—C≡N), isocyanato (—O—N⁺≡C⁻), thiocyanato (—S—C≡N), isothiocyanato (—S—N⁺≡C⁻), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₆-C₂₀ aryl)-substituted amino, C₂-C₂₄ alkylamido (—NH—(CO)-alkyl), C₅-C₂₀ arylamido (—NH—(CO)-aryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (CR═N(alkyl), where R=hydrogen, C₁-C₂₄ alkyl, aryl, alkaryl, aralkyl, etc.), arylimino (—CR═N(aryl), where R=hydrogen, alkyl, aryl, alkaryl, etc.), nitro (—NO₂), nitroso (NO), sulfonic acid (—SO₂—OH), sulfonato (—SO₂—O⁻), C₁-C₂₄ alkylsulfanyl (—S-alkyl; also termed “alkylthio”), C₅-C₂₀ arylsulfanyl (—S-aryl; also termed “arylthio”), C₁-C₂₄ alkylsulfinyl ((SO)-alkyl), C₅-C₂₀ arylsulfinyl ((SO)-aryl), C₁-C₂₄ alkylsulfonyl (—SO₂-alkyl), C₅-C₂₀ arylsulfonyl (SO₂-aryl), phosphono (—P(O)(OH)₂), phosphonato (—P(O)(O⁻)₂), phosphinato (—P(O)(O—)), phospho (—PO₂), phosphino (—PH₂), any with or without hetero atoms (e.g., N, O, S, or other) where the hetero atoms can be substituted (e.g., hetero atom substituted for carbon in chain or ring) for the carbons or in addition thereto (e.g., hetero atom added to carbon chain or ring) swapped, any including straight chains, any including branches, and any inducing rings, any being substituted or unsubstituted, derivatives thereof, and combinations thereof. The carbon chains can range from C₁-C₂₄, C₁-C₁₂, C₁-C₈, C₁-C₄, or C₁-C₂.

In some embodiments, the R¹ can be a hydrogen or an amine protecting group that is deprotectable upon treatment of an appropriate deprotection reagent. Amine protecting groups can be deprotectable by acid, such as the protecting group being tert-butyloxycarbonyl (Boc) group; or by a base, such as the protecting group being 9-a fluorenylmethyloxycarbonyl (Fmoc) group or trifluoroacetyl group; or by catalytic hydrogenation, such as the protecting group being a benzyl group; by photoirradiation, such as the protecting group being 2-nitrophenylethyl carbamate or 6-nitroveratryl carbamate or fluoride, such as a trimethylsilylethyloxycarbonyl (Teoc) group. Another option can include the leaving group being a 1,3-dithian-2-ylmethoxycarbonyl (Dmoc) group that can be deprotected under oxidative conditions. Other standard protecting groups may also be used. Also, the R¹ may be a leaving group that can be removed prior to coupling the amine to a substance.

In some embodiments, the R² can be a carbonyl protecting group. The carbonyl protecting group can include: alkyl esters (e.g., methyl ester) that can be removed by an acid or base; aryl esters (e.g., benzyl ester) that can be removed by hydrogenolysis; tert-butyl esters that can be removed by acid, base and some reductants; esters of 2,6-disubstituted phenols (e.g., (e.g. 2,6-dimethylphenol, 2,6-diisopropylphenol, 2,6-di-tert-butylphenol) that can be removed at room temperature by DBU-catalyzed methanolysis under high-pressure conditions; silyl esters that can be removed by acid, base and organometallic reagents; oxazoline that can be removed by hot acid or hot base at temperatures over 100° C.

In some embodiments, the R² can be a carbonyl leaving group, such as a hydrogen, hydroxyl, halide, alkoxy, anhydride, or amine. The halide (e.g., halogen ion) can be F, Cl, Br, or I. The alkoxy can be an alkyl as defined herein linked to an oxygen, where the oxygen is linked to the carbon of the carbonyl. The anhydride can include another carbonyl. The amine can be NH₂, or NHR³, wherein R³ can be a substituent as defined for the R group. Also, the R² may be a leaving group that can be removed prior to coupling the carbonyl to a substance.

In some embodiments, R⁹ and R¹⁰ can be the same or different. While R⁹ and R¹⁰ can be any chemical moiety, examples include hydrogen, alkyls (e.g., methyl, ethyl, propyl, butyl, etc.), cycloalkyls, cycloheterolaryls (e.g., with N hetero atom) and aryls (e.g., phenyl, or heteroaryls (e.g., pyrimidinyl, pyrrolidinyl, etc.).

The coumarin group can be any coumarin or coumarin derivative, which may be substituted or unsubstituted. However, as shown the coumarin group at least includes a first bond to the amine bonded to the R group and the L¹ linker arm and a second bond to the oxygen of the ester group, where the ester group is linked through the L² linker arm to the carbonyl of the second functional group. Often, the first bond is stable and not cleavable while the second bond can be within or linked to a reactive chemical moiety. Examples are provided herein. The coumarin can be an aminocoumarin having the amine as shown, where the amine can be on any possible carbon atom on the polycyclic ring of coumarin. Examples include the 3-aminocoumarin, 4-aminocoumarin, 7-aminocoumarin, or others. An example of the coumarin derivative is provided below:

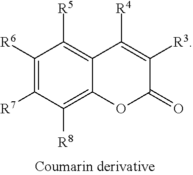

The coumarin derivative includes at least one of R³⁻⁸ being the amine (linker arm having L¹) of the aminocoumarin and at least one other of the R³⁻⁸ being linked to the ester group (linker arm having L²). The others of R³⁻⁸ may or may not be substituted, and when substituted can include a substituent as defined by the R group provided herein.

Accordingly, the “Coumarin” in Formula A can be the coumarin derivative, and thereby at least one of the R³⁻⁸ is the first linker arm:

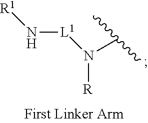

at least one other of the R³⁻⁸ is the second linker arm:

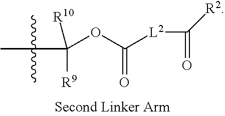

Accordingly, the coumarin derivative can include an aminocoumarin ester. The first linker arm can provide the amine of the aminocoumarin ester, and the second linker arm can provide the ester of the aminocoumarin ester. The amine and ester may be linked to any of the R³⁻⁸.

Accordingly, the other four R groups of coumarin derivative independently may be hydrogen or other substituent as defined for the R group. The R, R¹, R², R⁹, and R¹⁰ groups can independently be as defined herein.

In some embodiments, R⁹ and R¹⁰ are hydrogen so that the second linker arm has the following structure:

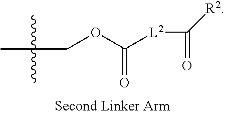

Some examples of the coumarin derivative, excluding the first linker arm and second linker arm, can include 3-aminocoumarin, 4-aminocoumarin, 7-aminocoumarin, umbelliferone (7-hydroxycoumarin), aesculetin (6,7-dihydroxycoumarin), herniarin (7-methoxycoumarin), psoralen and imperatorin. However, other coumarin derivatives can be used and lined to the first linker arm and the second linker arm as shown herein.

In some embodiments, the L¹ linker may be the same or different from the L² linker. The L¹ linker and L² linker can independently include straight aliphatics, branched aliphatics, cyclic aliphatics, substituted aliphatics, unsubstituted aliphatics, saturated aliphatics, unsaturated aliphatics, alkyleneoxides, polyalkyleneoxides, aromatics, polyaromatics, substituted aromatics, hetero-aromatics, amines, primary amines, secondary amines, tertiary amines, aliphatic amines, carbonyls, carboxyls, amides, esters, amino acids, polypeptides any with or without hetero atoms, derivatives thereof, substituted or unsubstituted, or combinations. In some aspects, the L¹ linker and L² linker can independently include C₁-C₂₄ alkyl, C₂-C₂₄ alkenyl, C₂-C₂₄ alkynyl, C₆-C₂₀ aryl, C₇-C₂₄ alkaryl, C₇-C₂₄ aralkyl, C₁-C₂₄ alkoxy, alkyleneoxides, polyalkyleneoxides, amino, mono- and di-(alkyl)-substituted amino, mono- and di-(aryl)-substituted amino, alkylamido, arylamido, imino, alkylimino, arylimino, nitro, nitroso, sulfo, sulfonato, alkylsulfanyl, arylsulfanyl, alkylsulfinyl, arylsulfinyl, alkylsulfonyl, arylsulfonyl, phosphono, phosphonato, phosphinato, phospho, phosphino, any with or without hetero atoms, any substituted or unsubstituted, derivatives thereof, and combinations thereof. The carbon chains, with or without hetero atoms, can include C₁-C₁₂, C₁-C₈, C₁-C₆, or C₁-C₄ chains, where any C can be substituted with a hetero atom, such as O, N, S, or P. Polypeptides or polymers can include any reasonable number of monomers, such as from 2-50, 2-30, 2-25, 2-20, 2-15, 2-10, or 2-5, or a single monomer thereof. The carbon chains can range from C₁-C₂₄, C₁-C₁₂, C₁-C₈, C₁-C₄, or C₁-C₂.

In some embodiments, examples of the L¹ linker can include alkyls, ethylene glycols, propylene glycols, ethers, esters, amides, oligoethylene glycols, polyethylene glycols, polypropylene glycols, or linker derived from amino-PEG-amine, or others.

Some examples of the amino-PEG-amine that be used as the L¹ linker are provided as follows; however, it should be recognized that the terminal amino and amine can be the nitrogen groups bounding the L¹ linker in the structures provided herein, which are incorporated into the structure with the appropriate bonding. Accordingly, instead of a primary amine, one amine group is bonded to the coumarin to be either a secondary amine when R is hydrogen, or a tertiary amine when R is not hydrogen. Similarly, the amine on the opposite end from the coumarin can be a primary or secondary amine with R¹ as defined herein. Specific examples can include (e.g., broadpharm.com) amino-PEG1-amine, amino-PEG2-amine, amino-PEG3-amine, amino-PEG4-amine, amino-PEG5-amine, amino-PEG6-amine, amino-PEG7-amine, amino-PEG8-amine, amino-PEG9-amine, amino-PEG10-amine, amino-PEG11-amine, and so on up to amino-PEG23-amine, or possibly more PEG monomers.

In some embodiments, examples of the L² linker can include alkyls, ethylene glycols, propylene glycols, ethers, esters, amides, oligoethylene glycols, polyethylene glycols, polypropylene glycols, or linker derived from Bis-PEG-acid, or others. Some examples of the Bis-PEG-acid that be used as the L² linker are provided as follows; however, it should be recognized that one of the terminal carboxyl groups can be bond the L² linker to the coumarin and form the ester group in the structures provided herein, which are incorporated into the structure with the appropriate bonding. Similarly, the carboxyl on the opposite end from the coumarin can be a modified with R² as defined herein. Specific examples can include (e.g., broadpharm.com) Bis-PEG11-acid, Bis-PEG2-acid, Bis-PEG3-acid, Bis-PEG4-acid, Bis-PEG5-acid, Bis-PEG6-acid, Bis-PEG7-acid, Bis-PEG8-acid, Bis-PEG9-acid, Bis-PEG10-acid, Bis-PEG11-acid, Bis-PEG12-acid, Bis-PEG13-acid, Bis-PEG14-acid, Bis-PEG15-acid, Bis-PEG16-acid, Bis-PEG17-acid, Bis-PEG18-acid, Bis-PEG19-acid, and so on up to Bis-PEG29-acid, or possibly more PEG monomers.

In some embodiments, the L¹ sub-linker includes alkyls, ethylene glycols, propylene glycols, ethers, esters, amides, oligoethylene glycols, polyethylene glycols, polypropylene glycols, or linker derived from amino-PEG-amine, or combinations thereof.

In some embodiments, the L² sub-linker includes alkyls, ethylene glycols, propylene glycols, ethers, esters, amides, oligoethylene glycols, polyethylene glycols, polypropylene glycols, or linker derived from Bis-PEG-acid, or combinations thereof.

In some embodiments, the capture entity is selected from the group of antibody, aptamer, peptide, protein, ligand, or receptor. The capture entity is configured or selected for the desired target, where the capture entity has an affinity and/or selectivity for the desired target. Capture entities and their respective targets are known and continue to be developed, and thereby any capture entity for a specific target can be used for the embodiments described herein.

In some embodiments, the substrate is selected from the group of well bottom, particle, bead, magnetic bead, porous member, non-porous member, solid member, microfluidic channel, microfluidic chamber, vessel, reservoir, or combination thereof.

In some embodiments, one of R¹ or R² is the substrate and the other of R¹ or R² is the capture entity.

In some embodiments, the L¹ in the first linker arm and/or the L² in the second linker arm may include a polyethylene glycol, having from 2 to 10 monomers or 2 to 25 monomers or 2 to 100 monomers. In some aspects, the L¹ is a PEG, and the L² may include oxopertanoic acid

In some embodiments, the heterobifunctional linker can include a structure of Formula B or derivative thereof:

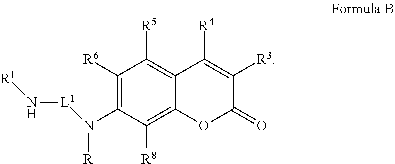

In Formula B, one of R³⁻⁶ or R⁸ is the second linker arm, and the rest of R³⁻⁶ or R⁸ are as defined for the R group provided herein.

In some embodiments, the heterobifunctional linker can include a structure of Formula C or derivative thereof:

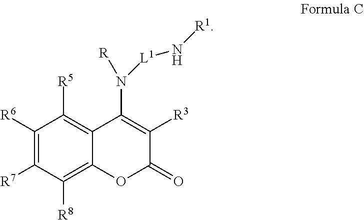

In Formula C, one of R³ or R⁵⁻⁸ is the second linker arm, and the rest of R³ or R⁵⁻⁸ are as defined for the R group provided herein.

In some embodiments, the heterobifunctional linker can include a structure of Formula D or derivative thereof:

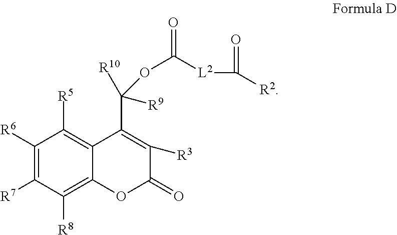

In Formula D, one of R³ or R⁵⁻⁸ is the first linker arm, and the rest of R³ or R⁵⁻⁸ are as defined for the R group provided herein.

In some embodiments, the heterobifunctional linker can include a structure of Formula E or derivative thereof:

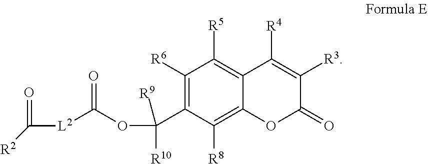

In Formula E, one of R³⁻⁶ or R⁸ is the first linker arm, and the rest of R³⁻⁶ or R⁸ are as defined for the R group provided herein.

In some embodiments, the heterobifunctional linker can include a structure of Formula F or derivative thereof:

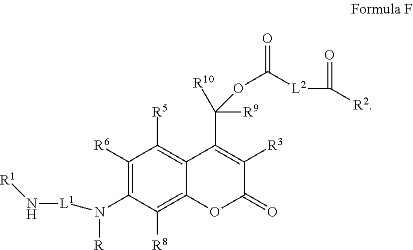

In Formula F, any of R³ or R⁵⁻⁶ or R⁸ are each independently as defined for the R group provided herein.

In some embodiments, the heterobifunctional linker can include a structure of Formula G or derivative thereof:

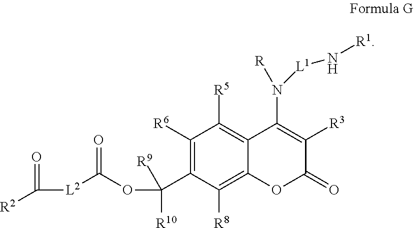

In Formula F, any of R³ or R⁵⁻⁶ or R⁸ are each independently as defined for the R group provided herein.

In some embodiments, the heterobifunctional linker can include a structure of Formula H or derivative thereof:

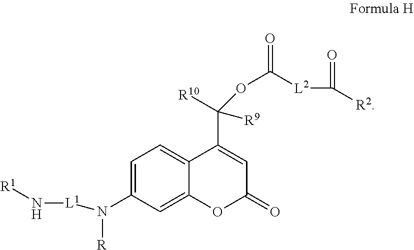

In some embodiments, the heterobifunctional linker can include a structure of Formula I or derivative thereof:

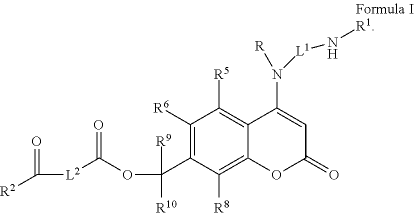

In some embodiments, the heterobifunctional linker can include a structure of Formula J or derivative thereof:

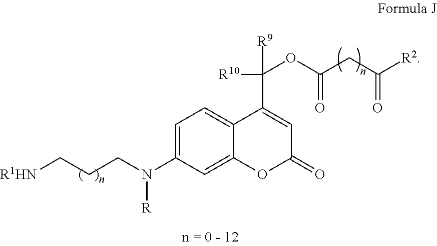

In Formula J, the R, R¹, R², R⁹ and/or R¹⁰ can be any R group substituent as defined herein, where specifically R¹ and/or R² can be the same as defined herein. In some aspects, the R, R⁹, and R¹⁰ groups can independently be hydrogen, methyl, ethyl, propyl, butyl, cyclopropyl, cyclobutyl, cyclohexyl, or other the like. In some aspects, R¹ can be an amine protecting group or amine leaving group. In some aspects, R² can be a carbonyl protecting group or carbonyl leaving group. Also, the “n” can be any integer, where examples can include any integer from 0-12, such as 0, 1, 2, 3, 4, 5, 6, 7, 8, 9, 10, 11, or 12. In some aspects, R¹ is an amine and R² is a hydroxyl. R⁹ and/or R¹⁰ can independently be any hydrogen, alkyl, cycloalkyl, or aryl, as described herein, which applies to any formula herein.

In some embodiments, the heterobifunctional linker can include a structure of Formula K or derivative thereof:

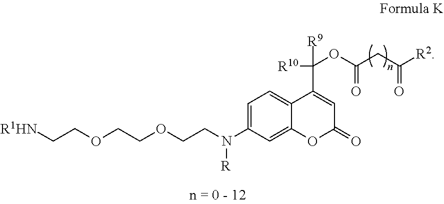

In Formula K, the R, R¹, R², R⁹, and/or R¹⁰ can be any R group substituent as defined herein, where specifically R¹ and/or R² can be the same as defined herein. In some aspects, the R, R⁹, and R¹⁰ group can independently be hydrogen, methyl, ethyl, propyl, butyl, cyclopropyl, cyclobutyl, cyclohexyl, or other the like. In some aspects, R¹ can be an amine protecting group or amine leaving group. In some aspects, R² can be a carbonyl protecting group or carbonyl leaving group. Also, the “n” can be any integer, where examples can include any integer from 0-12, such as 0, 1, 2, 3, 4, 5, 6, 7, 8, 9, 10, 11, or 12. In some aspects, R¹ is an amine and R² is a hydroxyl.

In some embodiments, the heterobifunctional linker can include a structure of Formula L or derivative thereof:

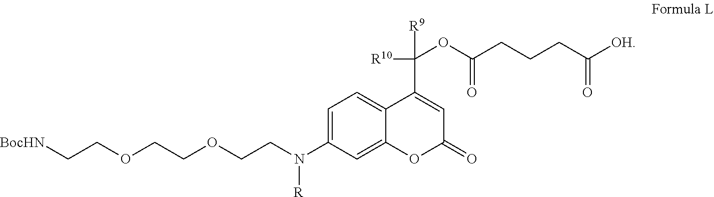

In Formula L, the R can be any R group substituent as defined herein. In some aspects, the R group can be hydrogen, methyl, ethyl, propyl, butyl, cyclopropyl, cyclobutyl, cyclohexyl, or other the like.

In some embodiments, the heterobifunctional linker can include a structure of Formula M or derivative thereof:

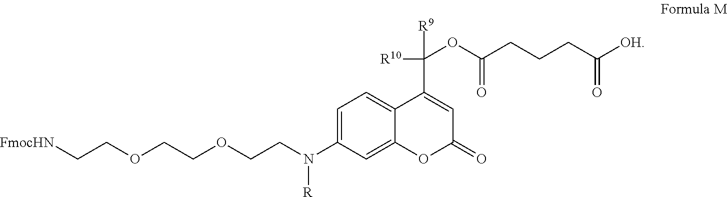

In Formula M, the R can be any R group substituent as defined herein. In some aspects, the R group can be hydrogen, methyl, ethyl, propyl, butyl, cyclopropyl, cyclobutyl, cyclohexyl, or other the like.

In some embodiments, the heterobifunctional linker can include a structure of Formula N or derivative thereof:

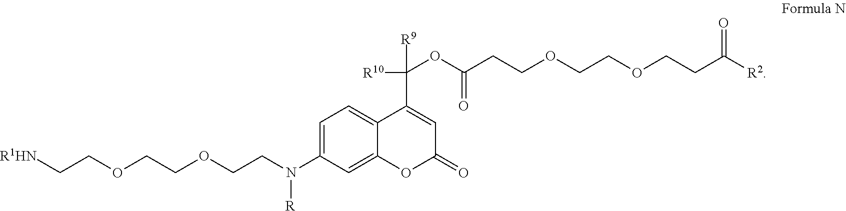

In Formula N, the R, R¹, and/or R² can be any R group substituent as defined herein, where specifically R¹ and/or R² can be the same as defined herein. In some aspects, the R group can be hydrogen, methyl, ethyl, propyl, butyl, cyclopropyl, cyclobutyl, cyclohexyl, or other the like. In some aspects, R¹ can be an amine protecting group or amine leaving group. In some aspects, R² can be a carbonyl protecting group or carbonyl leaving group. In some aspects, R¹ is an amine and R² is a hydroxyl.

In some embodiments, the heterobifunctional linker can include a structure of Formula O or derivative thereof:

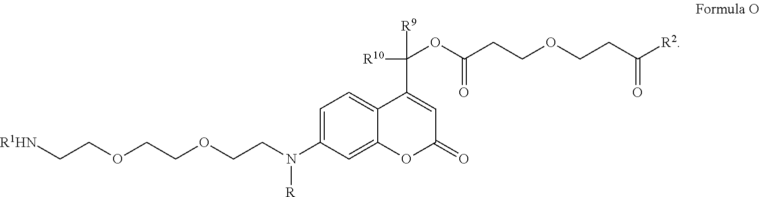

In some embodiments, the heterobifunctional linker can include a structure of Formula P or derivative thereof:

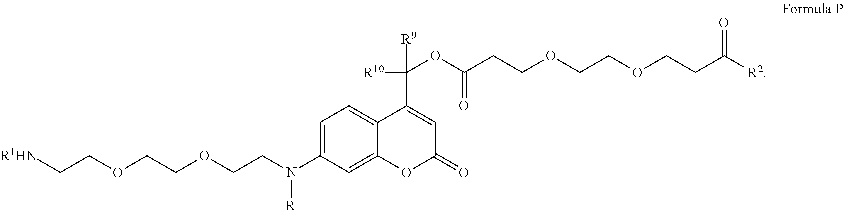

In some embodiments, the heterobifunctional linker can include a structure of Formula Q or derivative thereof:

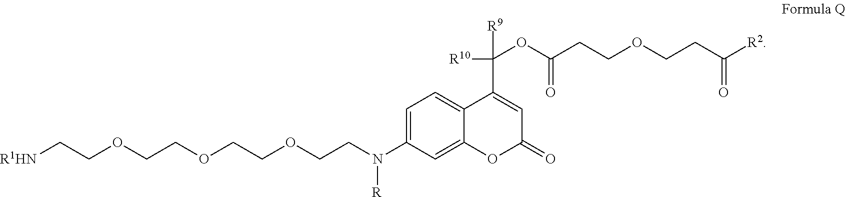

In some embodiments, the heterobifunctional linker can include a structure of Formula R or derivative thereof:

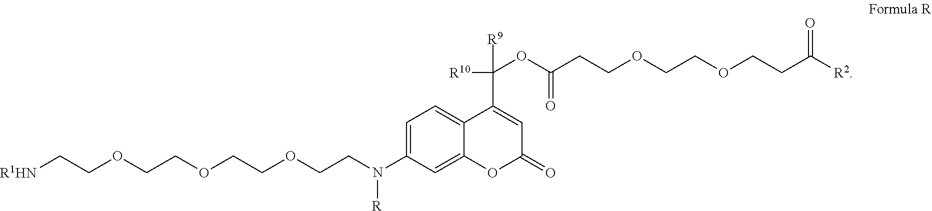

In any of the formulae provided herein, such as Formula A-R, the R, R¹, R², R⁹, and R¹⁰ groups are as defined herein. Additionally, the L¹ and L² groups are as defined herein.

The coumarin moiety and the ester moiety linked thereto cooperate to provide a photocleavable linker. Accordingly, light directed to the photocleavable linker can results in the ester breaking apart to leave the coumarin with a hydroxyl and the second linker arm having a carboxyl group. The configuration of the coumarin moiety, whether substituted or unsubstituted, can provide for different wavelengths of light that cause the photocleavage. In some example, the coumarin moiety can provide for the photocleavable moiety including a bond that is photocleavable with light having a wavelength greater than about 380 nm. In some aspects, the photocleavable moiety is cleavable with light having a wavelength greater than about 380 nm, greater than about 400 nm, greater than about 450 nm, and/or the wavelength can be greater than 495 nm and/or less than about 495 nm. The coumarin may be modified as described herein with appropriate substituents to tailor the wavelength of light that results in the photocleavage. For example, the photocleavable moiety can be configured to be cleavable with light having a wavelength from about 380 nm to about 570 nm (e.g., within the violet (380-450 nm)—blue (450-495 nm)—green (495-570 nm) wavelength spectrum). In some aspects, the photocleavable moiety is cleavable with light having a wavelength greater than UV-A light (e.g., greater than 380 nm, 390 nm, or 400 nm), wherein UV-A light can range from about 315 to 400 nm. In some aspects, the photocleavable linker is blue-light activated. In some aspects, the wavelength can be selected so that it is cleaved with visible light so as to avoid UV damage to the target biomarker.

As shown herein, the photocleavable moiety includes a coumarin group having a photostable amine and a photocleavable ester. In some aspects, the photocleavable moiety includes a coumarin group having a photostable amine on the first side or first end and a photocleavable ester on the second side or second end. In some examples, the photocleavable moiety includes a dialkylaminocoumaryl-alkyloxy, dialkylaminocoumaryl-4-alkyloxy, 7-dialkylaminocoumaryl-alkyloxy, or 7-dialkylaminocoumaryl-4-alkyloxy, dialkylaminocoumaryl-alkyloxy-oxopertanoic acid, 7-dialkylaminocoumaryl-4-alkyloxy-oxopertanoic acid, 7-(diethylamino)coumaryl-4-methoxy-oxopertanoic acid, wherein in each instance independently the alkyl is a C₁-C₁₂ alkyl, or C₁-C₈, or C₁-C₄, or C₁-C₂.

The photocleavable heterobifunctional linker can be prepared by a protocol that links the first linker arm and second linker arm to the coumarin. In some embodiments, the synthesis of the photocleavable heterobifunctional linker can include providing a coumarin having the structure provided herein, such as the coumarin derivative having R³-R⁸, where one of R³-R⁸ is a leaving group and another of R³-R⁸ is a protecting group and the rest of R³-R⁸ are each individually a chemical moiety. The leaving group can be reacted with a precursor of a first linker arm such that the first linker arm replaces the leaving group, where the first linker arm includes the R being a chemical moiety and the R¹ being an amine protecting group. The protecting group is then converted to an alcoholic group, which includes an alkyl portion and a hydroxyl portion. The alcohol group extending from the coumarin is then reacted with a precursor of a second linker arm so as to form an ester with the oxygen of the alcohol group in the second linker arm. The second linker arm can include the R² being a carbonyl protecting group. The amine protecting group and the carbonyl protecting groups can then be removed such that R¹ is hydrogen and R² is hydroxyl. However, the photocleavable heterobifunctional linker can be linked to a substrate and to a capture entity.

Accordingly, some embodiments include methods linking the linker to a substrate. Such linking methods can include deprotecting the amine protecting group to provide a primary amine. The, the method can include coupling the primary amine of the first linker arm with the substrate. For example, the primary amine and substrate are coupled through an EDC/NHS reaction.

In some embodiments, the methods can include linking the linker to a capture entity. Such linking methods can include deprotecting the carbonyl protecting group to provide a carboxylic acid. The method can include coupling the carboxylic acid to the capture entity. For example, the carboxylic acid and capture entity are coupled through an EDC/NHS reaction.

As shown herein, the first functional group, which can include the amine as defined herein, can be coupled to a first substance through a coupling reaction. For example, the coupling reaction can include an EDC/NHS reaction. The second functional group may include an appropriate blocking group during coupling of the first functional group to the first substance.

Also, the second functional group, which can include the carbonyl as defined herein, can be coupled to a second substance through a coupling reaction. For example, the coupling reaction can include an EDC/NHS reaction. The first functional group may include an appropriate blocking group during coupling of the second functional group to the second substance, or the first functional group can be coupled to the first substance prior to the second functional group being coupled to the second substance.

In some embodiments, a capture device includes the photocleavable heterobifunctional linker linked to a substrate. The other end of the heterobifunctional linker can be linked to a capture entity that can capture a substance. The substrate can include a chemical group that facilitate reaction with the first functional group. Also, the capture entity can include a chemical group that reacts with the second functional group. Once prepared, the capture entity can capture the targeted substance so that it is linked through the heterobifunctional linker to the substrate. Then, upon photocleavage, the targeted substance, while still associated with the second linker arm, or with the capture entity, is released from the substrate.

In some embodiments, the first linker arm of the heterobifunctional linker can be coupled to a substrate surface. The second linker arm can be coupled to a capture entity. In some aspects, the surface includes a cyclic olefin copolymer (COC). In some aspects, the surface includes UV/O₃-activated COC. In some aspects, the substrate is a particle, bead, porous member, non-porous member, or solid member or combination thereof. In some aspects, the first functional group is coupled to the surface through an amide coupling.

In some aspects, the capture entity can be configured for catching and/or releasing target substances, which can include biological substances. While some examples of biological substances that can be targeted by the capture entity can include circulating tumor cells (CTCs) or extracellular vesicles (e.g., exosomes), any other affinity selected biological substances can be the targets of the capture entity. Accordingly, the compounds and materials described herein can be used in any in vitro device or assay. The in vitro device or assay can use the compounds and materials for catching and/or releasing biological targets, such as biological markers, nucleic acids, aptamers, peptides, proteins, antibodies, and macro structures, such as circulating or non-attached cells of any type, CTCs and exosomes or others. The compounds and/or materials include the photocleavable bifunctional linker that can facilitate the catching and then releasing the target substance, such as described herein.

In some embodiments, a capture device can include the photocleavable bifunctional linker having a structure of Formula A, as presented herein, wherein: coumarin is any coumarin or coumarin derivative; R, R⁹, and R¹⁰ are each independently a chemical moiety; R¹ is a substrate; R² is a hydrogen, hydroxyl, halide, alkoxy, anhydride, amino, protecting group, leaving group, or capture entity; L¹ is a sub-linker; and L² is a sub-linker. In some aspects, the capture entity is selected from the group of antibody, aptamer, peptide, protein, ligand, or receptor. In some aspects, the substrate is selected from the group of well bottom, particle, bead, magnetic bead, porous member, non-porous member, solid member, or combination thereof. The substrate can include a cyclic olefin copolymer (COC), such as where the substrate includes a surface having UV/O₃-activated COC. In some aspects, the substrate includes a surface having exposed carbolic acid groups. In some aspects, the L¹ of the first linker arm is coupled to the substrate through an amide linkage. In some aspects, the L² is coupled to the capture entity through an amide linkage.

In some embodiments, the photocleavable heterobifunctional linker can be attached to a surface of a device, such as an in vitro diagnostic device or any other type of device. As such, the photocleavable heterobifunctional linker can facilitate linking of the captured elements to such as surface of an in vitro diagnostic device for use in assays, such as with liquid biopsy assays. When intact, the photocleavable heterobifunctional linker can link the captured element to the surface. When cleaved by photocleavage, the photocleavable heterobifunctional linker can release the captured element from the surface. As such, the photocleavable heterobifunctional linker can facilitate liquid biopsy assays.

In some embodiments, the invention includes a blue-light activated photocleavable heterobifunctional linker having a central coumarin group, a terminal amine, and a terminal carbonyl (e.g., carboxyl) group to selectively capture and release biomarkers or other biological substances. The heterobifunctional aspect of the linker allows binding to a surface on one end and binding of a selective capture entity (e.g., antibody, aptamer, ligand, receptor, or other capture moiety) on the other end. The binding of the ends of the linker can include two EDC/NHS reactions that are performed sequentially with appropriate protecting/leaving groups to protect the end not being reacted in the first step. The coumarin photocleavable group cleaves via non-invasive blue light, thereby reducing the damage to cells and genetic material typical of UV-A cleavage methods. The coumarin linker's strong cleavage quantum efficiency enables rapid biomarker release without the need for costly reagents. The photocleavable heterobifunctional linker does not require capture element modifications that may result in capture element loss of activity.

While coumarins are used herein as chromophores for the photocleavable moiety, any chromophore may be used. As such, the selection of the chromophore can determine the wavelength of light to cause the cleaving.

The photocleavable heterobifunctional linker allows for the selective catch and release of biomarkers, such as liquid biopsy markers, such as those described herein. the linker provides for an efficient attachment to a carboxylated surface of a first substance (e.g., substrate surface) at one end of the linker and to capture entities (e.g., antibodies, aptamers, and others described herein). The capture entity can then selectively capture target substances from a fluid medium. The application of the light cleaves the linker to release the capture entity. This allows for selection and enrichment of the target substance.

In some embodiments, a method of capturing a target substance can include providing the capture device of one of the embodiments, wherein the R² is the capture entity, and contacting a target substance to the capture moiety such that the target substance is captured. The capture entity can be selected from the group of antibody, aptamer, ligand, or receptor. The substrate can be selected from the group of well bottom, particle, bead, magnetic bead, porous member, non-porous member, solid member, or combination thereof. The target substance can be selected from a circulating cell, nucleic acid, peptide, protein, extracellular vesicle, exosome, or analyte.

In some embodiments, a method of releasing a captured target substance can include providing the capture device of one of the embodiments, where the R² is the capture entity having a target substance associated therewith, and irradiating the photocleavable heterobifunctional linker with light that cleaves the linker, thereby releasing the target substance from the substrate. In some aspects, the irradiating is with light having a wavelength greater than about 380 nm. In some aspects, the irradiating is with light having a wavelength from about 380 nm to about 570 nm.

In some embodiments, the linker can be used in liquid biopsies to detect the target biomarker. Accordingly, a body fluid, such as blood, urine, or other, can be collected and contacted to the biopsy device that has the linker attached to a substrate at one end and to a capture entity at the other end. After the target biomarker is captured, light can be used to irradiate the linker to cleave the linker and release the arm having the target biomarker. The target biomarker can then be assayed as common in liquid biopsy protocols.

### EXPERIMENTAL

Photocleavable Heterobifunctional Linker Synthesis

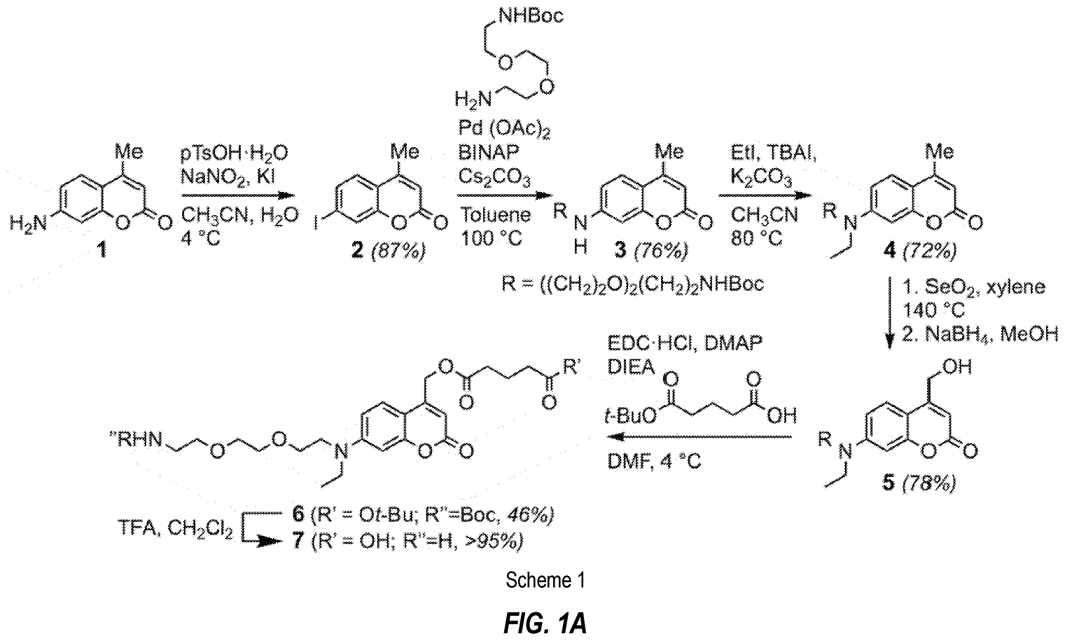

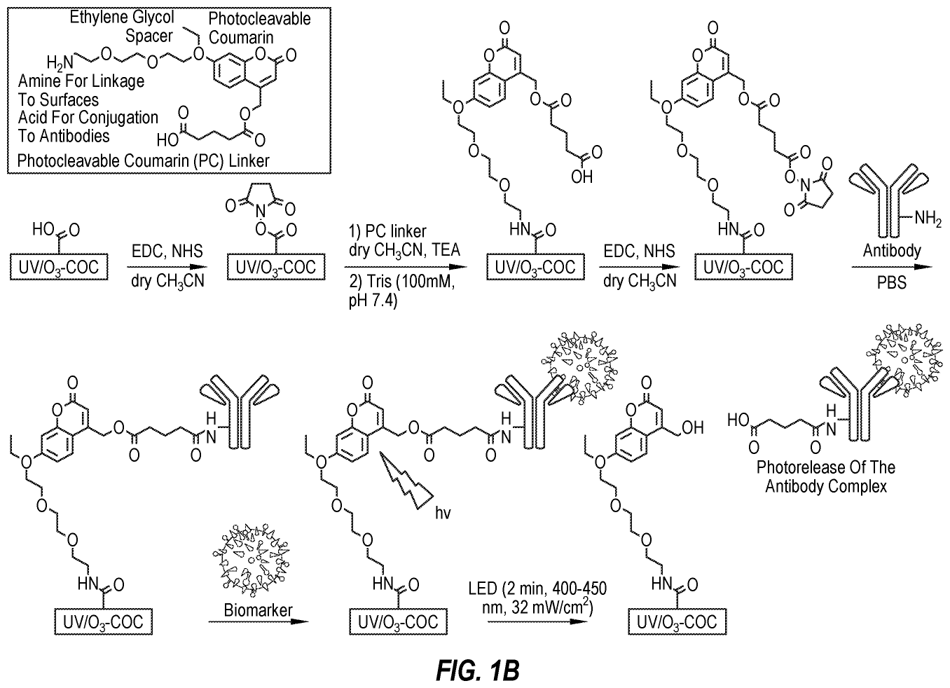

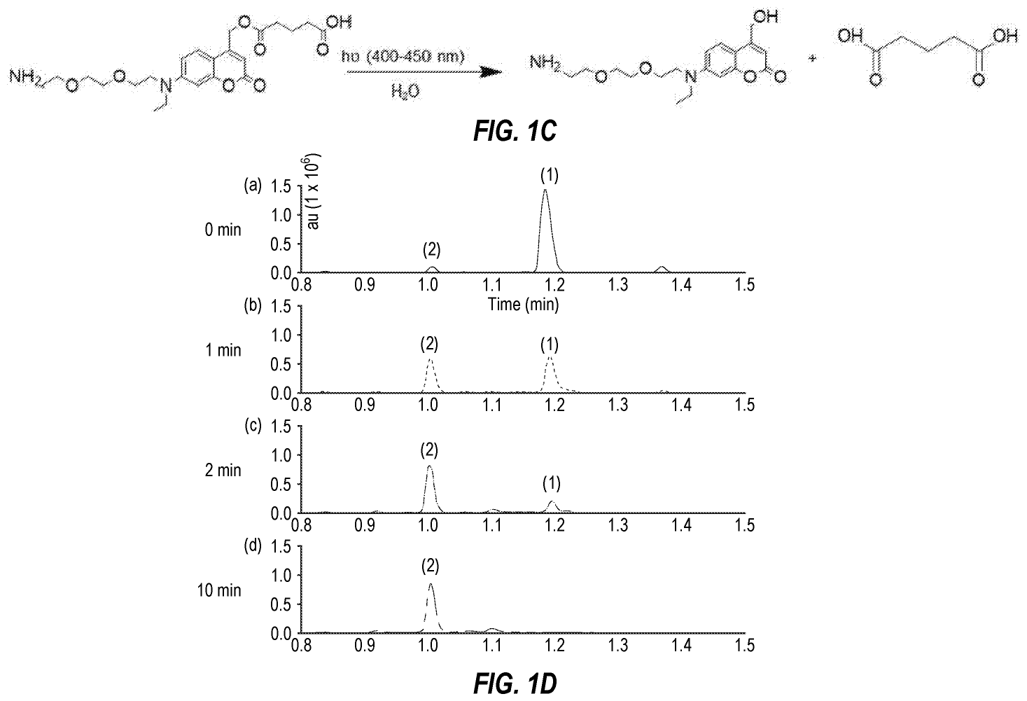

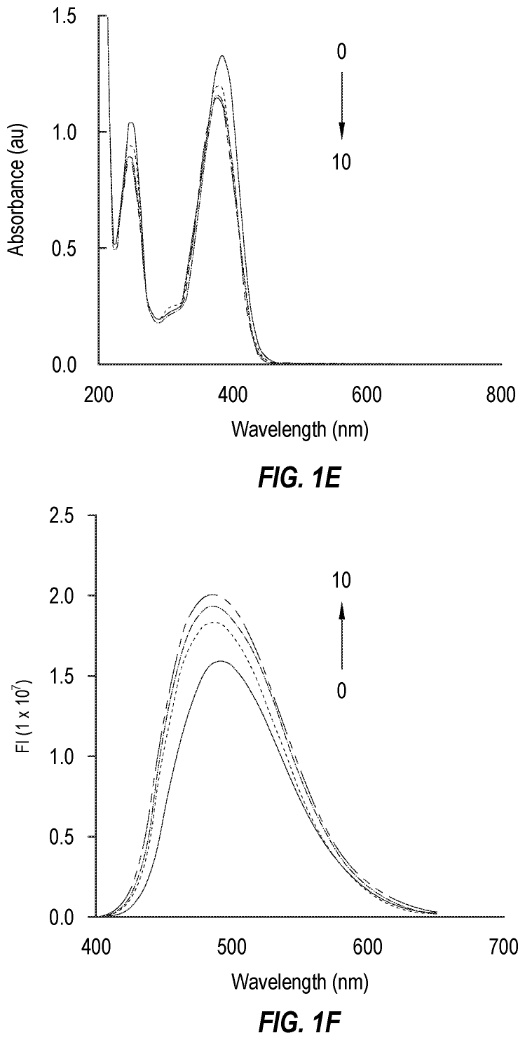

The photocleavable heterobifunctional linker was synthesized as shown in Scheme 1 of FIG. 1A. Scheme 1 includes the following reaction protocol. Briefly, 7-amino-4-methyl-2H-chromen-2-one (Compound 1) (710 mg, 2.48 mmol, 1 equiv.), and p-toluenesulfonic acid monohydrate (1.63 g, 8.56 mmol, 3.0 equiv.) were weighed in a single necked round bottom flask (100 mL) and suspended in acetonitrile:water (6 mL, 1:1). The suspension was cooled to 4° C. for 5 min and treated dropwise with sodium nitrite (390 mg, 5.70 mmol, 2.0 equiv.) and potassium iodide (1.18 g, 7.13 mmol, 2.5 equiv.) in water (4 mL). Vigorous effervescence was observed. After the addition of this reagent was complete, the reaction mixture was stirred at 4° C. for an additional 10 min and then stirred at room temperature (23° C.) for 3-4 h. Reaction progress was monitored by TLC and upon completion, saturated aqueous sodium bicarbonate was added to adjust the pH to 9. The reaction mixture was then diluted with ethyl acetate (˜250 mL), and the organic layer was extracted with water (2×50 mL) and saturated aqueous sodium thiosulfate (50 mL). The organic layer was dried over anhydrous sodium sulfate, filtered, and concentrated to dryness. The residue was re-dissolved in dichloromethane and purified by silica gel chromatography using hexane and ethyl acetate to elute Compound 2 (710 mg, 87% yield) as a colorless solid. ¹H NMR (400 MHz, Chloroform-d) δ 7.73-7.69 (m, 1H), 7.65-7.60 (m, 1H), 7.30 (d, J=8.3 Hz, 1H), 6.31 (d, J=1.5 Hz, 1H), 2.42 (d, J=1.2 Hz, 3H); ¹³C NMR (101 MHz, Chloroform-d) δ 160.0, 153.6, 152.0, 133.6, 126.3, 125.7, 119.7, 115.8, 97.3, 18.7.

The iodide Compound 2 (120 mg, 0.42 mmol, 1.0 equiv.), amine (124 mg, 0.5 mmol, 1.2 equiv.), Pd(OAc)2 (9.5 mg, 0.042 mmol, 0.1 equiv.), Xantphos (36.4 mg, 0.063 mmol, 0.15 equiv.) and Cs₂CO₃ (342 mg, 1.05, 2.5 equiv.) were weighed in a Biotage microwave reaction vial in a glove box. The mixture was treated with toluene (6 mL), and the vial was sealed and removed from the glove box. The reaction mixture was heated in an oil bath at 100° C. for 6 h. The crude mixture was then cooled to room temperature and purified by silica gel chromatography using hexane and ethyl acetate to elute Compound 3 (129 mg, 76% yield) as a viscous oil. ¹H NMR (400 MHz, Chloroform-d) δ 7.36 (d, J=8.6 Hz, 1H), 6.59 (s, 1H), 6.50 (s, 1H), 5.99 (s, 1H), 4.99 (s, 1H), 3.73 (t, J=5.1 Hz, 2H), 3.65 (s, 4H), 3.56 (t, J=5.2 Hz, 2H), 3.38 (t, J=5.1 Hz, 2H), 3.33 (s, 2H), 2.34 (d, J=1.0 Hz, 3H), 1.44 (s, 9H); ¹³C NMR (101 MHz, CDCl₃) δ 162.0, 156.1, 156.1, 153.1, 151.5, 125.6, 110.9, 110.8, 109.6, 98.3, 79.5, 70.5, 70.3, 70.3, 69.2, 43.2, 40.5, 28.5, 18.6; HRMS calcd. for C₂₁H₃₁N₂O₆+: 407.2182; Found: 407.2187.

The amine Compound 3 (35 mg, 0.086 mmol, 1.0 equiv.), tetrabutylammonium iodide (31.7 mg, 0.086 mmol, 1.0 equiv.) and potassium carbonate (41.5 mg, 0.30 mmol, 3.5 equiv.) were weighed in a flame-dried, Ar-flushed Biotage microwave reaction vial. The mixture was treated with anhydrous acetonitrile (3 mL) and iodoethane (0.027 mL, 0.344 mmol, 4.0 equiv.). The vial was sealed and heated in an oil bath at 80° C. for 16 h. After 16 h, the reaction mixture was treated with additional 2.0 equiv. of iodoethane and heated for 18-20 h. Progress of the reaction was monitored by TLC, and upon completion, the reaction was diluted with ethyl acetate (25 mL) and transferred to a separatory funnel. The organic layer was washed with water (2×10 mL), dried over sodium sulfate, and concentrated to dryness. The residue was re-dissolved in dichloromethane and purified by silica gel chromatography using hexane and ethyl acetate to elute Compound 4 (27 mg, 72% yield) as a viscous oil. ¹H NMR (400 MHz, Chloroform-d) δ 7.39 (d, J=8.9 Hz, 1H), 6.67 (dd, J=8.9, 2.2 Hz, 1H), 6.56 (d, J=2.3 Hz, 1H), 5.97 (d, J=1.0 Hz, 1H), 4.96 (s, 1H), 3.69-3.64 (m, 2H), 3.62-3.55 (m, 6H), 3.53 (t, J=5.2 Hz, 2H), 3.48 (q, J=7.1 Hz, 2H), 3.30 (s, 2H), 2.34 (d, J=1.0 Hz, 3H), 1.44 (s, 9H), 1.20 (t, J=7.1 Hz, 3H); ¹³C NMR (101 MHz, CDCl₃) δ 162.2, 156.1, 156.0, 152.9, 150.7, 125.6, 109.9, 109.4, 108.9, 98.4, 79.4, 70.9, 70.4, 70.4, 68.7, 50.5, 46.2, 40.5, 28.6, 18.6, 12.1; HRMS calcd. for C₂₃H₃₅N₂O₆+: 435.2495; Found: 435.2487.

The Compound 4 (54 mg, 0.12 mmol, 1.0 equiv.) and selenium dioxide (27.5 mg, 0.24 mmol, 2.0 equiv.) were weighed in a flame-dried, Ar-flushed, single-necked round bottom flask (25 mL) equipped with a water-cooled condenser. The mixture was treated with p-xylene (5 mL) and heated in an oil bath at 140° C. for 24 h. The crude reaction mixture was cooled to room temperature and concentrated to dryness. Formation of the intermediate aldehyde was verified by ¹H NMR in chloroform-d. The crude mixture was then re-dissolved in methanol (1 mL), treated with sodium borohydride (14.0 mL, 0.37 mmol, 3.0 equiv.), and stirred at room temperature (23° C.) for 3 h. The progress of the reaction was monitored by TLC, and upon completion, the reaction mixture was concentrated to dryness. The crude mixture was re-dissolved in dichloromethane and purified by silica gel chromatography using hexane and ethyl acetate to elute Compound 5 (44 mg, 78% yield) as a viscous oil. ¹H NMR (400 MHz, Chloroform-d) δ 7.31 (d, J=9.0 Hz, 1H), 6.60 (dd, J=9.0, 2.5 Hz, 1H), 6.52 (d, J=2.5 Hz, 1H), 6.27 (s, 1H), 5.00 (s, 1H), 4.80 (s, 2H), 3.64 (t, J=5.8 Hz, 2H), 3.57 (d, J=13.2 Hz, 6H), 3.52-3.42 (m, 4H), 3.27 (q, J=5.0 Hz, 2H), 1.42 (s, 9H), 1.18 (t, J=7.1 Hz, 3H); ¹³C NMR (101 MHz, CDCl₃) δ 162.6, 156.2, 156.1, 155.0, 150.7, 124.6, 109.0, 107.0, 106.1, 98.3, 79.5, 70.9, 70.4 (2 overlapping carbons), 68.7, 61.0, 50.3, 46.0, 40.4, 28.5, 12.1; HRMS calcd. for C₂₃H₃₅N₂O₇⁺: 451.2444; Found: 451.2455.

5-(tert-butoxy)-5-oxopentanoic acid (20 mg, 107 μmol, 1.1 equiv.), EDC-HCl (22.5 mg, 117 μmol, 1.2 equiv.) and dimethylaminopyridine (1.2 mg, 9.7 μmol, 0.1 equiv.) were weighed in a flame-dried, Ar-flushed microwave vial. The mixture was cooled to 0° C. and sequentially treated with anhydrous DMF (0.5 mL) and DIEA (35 μL, 195 μmol, 2.0 equiv.) and stirred for 30 min. The reaction mixture was then treated dropwise with alcohol Compound 5 (44 mg, 97 μmol, 1.0 equiv.) as a solution in DMF (1.0 mL). The reaction mixture was stirred at 0° C. for 10 min and then warmed to room temperature (23° C.). The reaction mixture was stirred for an additional 16-18 h. Upon completion, the reaction mixture was diluted with ethyl acetate (25 mL) and transferred to a separatory funnel. The organic layer was washed with water (2×10 mL), dried over sodium sulfate, and concentrated to dryness. The residue was re-dissolved in dichloromethane and purified by silica gel chromatography using hexane and ethyl acetate to elute Compound 6 (28 mg, 46% yield) as a viscous oil. ¹H NMR (600 MHz, Chloroform-d) δ 7.29 (d, J=9.0 Hz, 1H), 6.61 (dd, J=9.0, 2.6 Hz, 1H), 6.55 (d, J=2.5 Hz, 1H), 6.14 (s, 1H), 5.22 (d, J=1.4 Hz, 2H), 4.94 (s, 1H), 3.66 (t, J=6.0 Hz, 2H), 3.59 (d, J=17.0 Hz, 6H), 3.53 (t, J=5.3 Hz, 2H), 3.48 (q, J=7.1 Hz, 2H), 3.31 (q, J=5.5 Hz, 2H), 2.50 (t, J=7.5 Hz, 2H), 2.31 (t, J=7.3 Hz, 2H), 1.97 (m, 2H), 1.44 (d, J=4.4 Hz, 18H), 1.20 (t, J=7.0 Hz, 3H); ¹³C NMR (151 MHz, Chloroform-d) δ 172.5, 172.2, 161.9, 156.3, 156.1, 151.0, 149.5, 124.5, 109.0, 107.0, 106.5, 98.3, 80.7, 79.4, 70.9, 70.4, 70.4, 68.7, 61.4, 50.4, 46.0, 40.4, 34.6, 33.3, 28.6, 28.3, 20.3, 12.1; HRMS calcd. for C₃₂H₄₉N₂O₁₀⁺: 621.3387; Found: 621.3379.

The ester Compound 6 (25 mg, 40.2 μmol, 1.0 equiv.) was dissolved in dichloromethane (0.7 mL) and treated with TFA (0.3 mL) at room temperature (23° C.). The reaction mixture was stirred for 1 h, and progress of the reaction was monitored by TLC. Upon completion, the reaction mixture was concentrated to dryness, and the excess TFA was removed azeotropically using toluene. The residue was re-dissolved in DMSO and purified by reverse phase chromatography using water and acetonitrile (both containing 0.1% TFA). Dissolution in water and lyophilization yielded pure Compound 7 (18 mg, >95% yield) as a viscous oil. ¹H NMR (400 MHz, Methanol-d₄) δ 7.49 (d, J=9.1 Hz, 1H), 6.80 (dd, J=9.1, 2.6 Hz, 1H), 6.69 (d, J=2.6 Hz, 1H), 6.12-6.06 (m, 1H), 5.32 (d, J=1.3 Hz, 2H), 3.98 (s, 1H), 3.75-3.61 (m, 11H), 3.55 (q, J=7.0 Hz, 2H), 3.15-3.08 (m, 2H), 2.56 (t, J=7.4 Hz, 2H), 2.39 (t, J=7.3 Hz, 2H), 2.00-1.91 (m, 2H), 1.21 (t, J=7.0 Hz, 3H); ¹³C NMR (126 MHz, Methanol-d₄) δ 176.7, 175.5, 165.0, 158.7, 157.2, 152.6, 125.8, 110.6, 107.8, 105.2, 98.7, 71.8, 71.4, 70.6, 69.9, 60.8, 51.1, 46.6, 40.3, 36.1, 34.0, 22.3, 12.2; HRMS calcd. for C₂₃H₃₃N₂O₈⁺: 465.2238; Found: 465.2231.

The linker was purified by HPLC (87.5% purity) using a Waters Acquity HPLC equipped with an LCT Premier TOF MS system, Acquity BEH C-18 and 1.7 m, 2.1×50 mm column running an ammonium hydroxide (pH 9.8) and methanol gradient (5-95% organic in 2 min at 0.6 mL/min). The purified linker was confirmed by NMR before being dried, resuspended in acetonitrile (ACN) for aliquoting and drying under an N₂ stream, and stored at −80° C.

The synthesized PC linker was dissolved in 1×PBS (2.1 μM) and exposed to visible light (400-450 nm, 34±4 mW/cm2). Samples (50 μL) were withdrawn after 1 min, 2 min and 10 min light irradiation and analyzed by UPLC/HRMS (Waters Acquity UPLC with a photodiode array UV detector and an LCT Premiere TOF mass spectrometer). E the mobile phase consisted of a gradient of water/acetonitrile (95:5 to 0:100 containing 0.05% TFA) over 2.7 min. The column consisted of a Waters Acquity Atlantis T3 2.1×50 mm, 1.7 μm column operated at a flow rate of 0.6 mL/min. The wavelength of detection was 247 nm and the volume injected onto the column was 2 μL.

Surface Characterization

Solvent treatment for UV/O₃-activated COC (cyclic olefin copolymer) —COOH surfaces was performed. COC plates (6013S-04) were diced into 1″ squares, cleaned of debris, rinsed with 10% Micro-90, IPA, and nanopure water, and dried at 60° C. for >1 h. Substrates were UV/O₃-activated (16.1 min, 22 mW/cm2 measured at 254 nm) and either left in air or immersed in 100 mM MES buffer (pH=4.8) or anhydrous solvents—ACN, dimethylformamide (DMF), or dichloromethane (DCM)—for 2 h. Treated substrates were rinsed with copious amounts of nanopure water and N₂ dried.

Water contact angles (WCA) were measured by dispensing 2.0 μL of nanopure water onto the appropriate substrate using a VCA Optima instrument (AST Products) and the manufacturer's software. Each substrate was measured in triplicate with at least 3 measurements per substrate.

Carboxylic acid (—COOH) surface densities were measured via toluidine blue 0 (TBO). The TBO assay was performed by attaching an in situ incubation chamber to the substrate's surface and filling with 0.1% (w/v) TBO in 50 mM carbonate buffer (pH=10.5). After 15 min, the substrate was submersed in the same buffer for 15 min then air dried. TBO was desorbed using 40% acetic acid (d=1.0196 g/mL) and collected in a pre-weighed microfuge tube. TBO concentrations were determined with either a Shimadzu UV-1280 UV/Vis spectrophotometer or a BioTek Synergy H4 Hybrid plate reader against a calibration curve and a 40% acetic acid blank.

Attenuated total reflectance Fourier transform infrared spectroscopy (ATR-FTIR) spectra were acquired using a Shimadzu IRAffinity-1S equipped with a Specac Quest ZnSe ATR accessory. Each scan (340-4700 cm⁻¹) was averaged 45 times and processed by a 3-point baseline correction (1500, 2000, and 4000 cm⁻¹) before integrating peak areas for carbonyls (1650-1850 cm⁻¹) and hydroxyls (3200-3700 cm⁻¹).

Cy5-Oligonucleotide Immobilization

Surfaces were UV/O₃-activated directly or microfluidic devices (thermal fusion bonded at 134° C., 1 h) were UV/O₃-activated through the cover plate. Surfaces or devices were then activated with EDC (20 mg/mL) and NHS (2 mg/mL), either solubilized in 100 mM MES buffer (pH=4.8) by pipetting in anhydrous ACN by vortexing for 25 min at room temperature. After air drying, ssDNA oligonucleotides with 5′-NH2, 3′-Cy5 modifications were infused at 40 μM concentration in PBS. The reaction was carried out for 2 h at room temperature before rinsing with 0.1% SDS then PBS. Imaging was conducted with a Zeiss Axiovert 200M microscope using a 10× objective (0.3 NA, Plan NeoFluar), an XBO 75 lamp, Cy5 filter set (Omega Optical), a Cascade 1K EMCCD (Photometrics) camera, and a MAC 5000 stage (Ludl Electronic Products), all of which were computer-controlled via Micro-Manager. Collected images were background subtracted, measured, and intensity-scaled for display in ImageJ. Flat substrates, beads, or microfluidic devices can be used for substrates.

Photocleavable Heterobifunctional Linker Immobilization and Labeling

Devices were UV/O₃-activated, EDC/NHS-activated in ACN, air-dried, then infused with PC linker resuspended in ACN. The devices were immediately wrapped in Rubylith© film to protect the linker from premature cleavage by exposure to ambient light. After 2 h, the linker was displaced by air, and the device was rinsed with 100 μL ACN before quenching free NHS esters with 100 mM tris buffer (pH=7.2) for 30 min followed by removing the tris buffer with air before washing with ACN. The immobilized PC was labeled with Cy5-oligonucleotide using EDC/NHS coupling in ACN, as described above for initial testing of PC surface immobilization and subsequent release.

Photocleavable Heterobifunctional Photoexposure

The linker was irradiated using an LED (M420L3, ThorLabs) outputting 885 mW light from 385-470 nm (λₘₐₓ=412 nm) and filtered through a 400 nm longpass colored glass filter (Edmund Optics). The LED's innate divergence (60°) was used to illuminate a 90 mm diameter spot at a 24 mm distance. The power distribution was measured with an 18 mm×18 mm power sensor (ThorLabs) rastered beneath the LED spot. These measurements were then fit with a 2D Gaussian and integrated over the device's surface area using Matlab. For photoexposure, the LED was mounted to a polished aluminum chamber and triggered using an analog LED driver (Thorlabs) and a custom electronic timer. After photo-exposure, cleaved Cy5-oligonucleotides were collected by infusing 100 μL PBS and recovering the effluent in a pre-weighed microfuge tube. Fluorometric measurements were made using a Jobin-Yvon Fluorolog 3 fluorometer (λₑₓ=642 nm, λₑₘ=664 nm).

Cy5 Reporters

For releasing Cy5 reporters immobilized via the PC linker, devices were first imaged by fluorescence microscopy as described above. Devices were then exposed to LED light for 1 min. Released Cy5 reporters were removed from the device by infusing PBS, collected into a pre-weighed microcentrifuge tube, and quantified against a calibration curve by fluorometry (Jobin-Yvon Fluorolog 3, λex=642 nm, λem=664 nm). Devices were imaged by microscopy again after the photocleavage process was repeated for an additional 1 min (2 min total exposure) and a final 8 min LED exposure (10 min total) to investigate the efficiency of photocleavage as a function of dose.

Cell Culture, Enrichment, and Release

SKBR3, MCF7, and HS578T (breast cancer, adherent) cell lines were cultured at 37° C. under a 5% CO₂ atmosphere in 1× McCoy's 5 A/10% FBS, 1× MEM alpha/10% FBS and 1.7 μM human insulin, or DMEM/10% FBS/and 1.7 μM bovine insulin, respectively. Cells were harvested for experiments using TrypLE express reagent (5 min) and were centrifuged (300 g, 10 min) and resuspended in ice cold PBS. De-identified blood samples from healthy donors were provided by the KU Cancer Center's Biospecimen Repository Core Facility (BRCF) under the repository's IRB approved protocol (HSC #5929).

Prior to sample infusion, CTC devices modified with the PC linker and anti-EpCAM Abs were infused with 2 mL of 0.5% BSA/PBS at 50 μL/min to remove unbound Abs and block the surface to minimize nonspecific adsorption. SKBR3 cells were pre-stained with Hoechst 33342 (40 μg/mL, 15 min, RT), resuspended in PBS, then spiked into a 1 mL blood sample (69-269 SKBR3 cells/mL). The spiked blood was loaded into a 1-3 mL syringe (BD) and hydrodynamically infused through two devices in series at a flow rate of 25 μL/min (2 mm/s linear velocity). After blood processing, nonspecifically bound cells were removed by rinsing with 1 mL of 0.5% BSA/PBS at a flow rate of 50 μL/min (4 mm/s linear velocity). All cells were stained with SYTO 82 nucleic acid dye (5 μM, infused at 25 μL/min and incubated for 15 min). Excess dye was removed with 0.5% BSA/PBS (50 μL/min, 100 μL). Devices were exposed to the LED system (2 min, 32±4 mW cm², described above), and released cells rinsed with 0.5% BSA/PBS (50 μL/min, 250 μL) and collected into a flat bottom 96 well plate for fluorescence microscopy (DAPI and Cy3 filters). Additionally, the microfluidic device was manually scanned to enumerate cells that were not released.

SKBR3 cells were identified as positive for both Hoechst 33342 and SYTO 82, whereas nonspecific leukocytes were positive for SYTO 82 only. Purity was calculated as the ratio of SKBR3 cells to total cell count (SKBR3 cells+leukocytes). Release efficiency was calculated as the ratio of released cells to the total cell count (released cells+cells on-chip). Capture efficiency was determined by self-referencing, where the SKBR3 cells captured in the first device was divided by the total cell count (first device+second device). Mouse IgG2A isotype control antibody was immobilized through the PC linker to evaluate nonspecifically bound SKBR3 cells. This was undertaken to gauge the release efficiency of MCF7 and Hs578T cells spiked into PBS buffer using Hoechst 33342 staining only. In the case of Hs578T cell experiments, anti-FAPa monoclonal antibodies were used.

Cell Viability, Cultivation and Oxidative DNA/NRA Damage

SKBR3 cells were spiked into PBS and affinity-enriched with anti-EpCAM Abs. Cells were released by 2 min LED exposure, collected into a 96 well plate, and stained for viability using calcein AM and ethidium homodimer I (LIVE/DEAD Cell Imaging Kit) for 15 min at room temperature. The plate was centrifuged (300 rcf, 5 min), and staining reagents were aspirated and replaced with PBS for fluorescence microscopy. Viability measurements were taken from ˜100 released cells (for other cell lines, the release step was omitted, and cells were directly exposed in a 96 well plate—for these viability measurements, several thousand cells were averaged). For monitoring cell cultivation after release, SKBR3 cells (180 cells) were seeded into the experiment and were then cultured (as described above) for up to 4 days.

LED-induced DNA/RNA damage was determined by measuring the oxidative product of DNA/RNA, 8-oxo guanine (8-oxo-G). Hs578T cells were grown in 35 mm diameter tissue culture dishes (Fisher Scientific) until ˜80% confluency. The cells were washed with ice cold PBS, covered with 1 mL ice cold PBS, and the culture dish was irradiated in an ice bath in the LED exposure system for 2 min (18.47 J). In a control experiment, the cells were placed in the exposure system for 2 min without irradiation. To allow for comparison of LED exposure and UV exposure, this process was repeated with an equivalent dosage of UV light (18.47 J) using a UVP CL-1000 crosslinker chamber (Analytik Jena). Results from UV and LED irradiation were compared to results obtained from H₂O₂ treated cells. Briefly, the cells were washed twice with ice cold PBS, incubated in 10 mL H₂O₂ (300 μM in 1×PBS) for 30 min at 37° C. and 5% CO₂, then washed twice with ice cold PBS.

DNA or RNA was immediately extracted following irradiation using Zymo Quick-DNA and Direct-zol RNA isolation kits according to the manufacturer's protocol. Extracted DNA/RNA was quantified by UV-Vis (Shimadzu BioSpec-nano) and High Sensitivity RNA or Genomic DNA Tapestation (Agilent) and diluted to 80 ng/μL, and 6 μg substrate was digested into mononucleotides using 18 mU phosphodiesterase I, 15 U benzonase nuclease, and 12 U alkaline phosphatase in NEBuffer 2.1. 8-oxoG quantification was performed in triplicate using a DNA Damage Competitive ELISA Kit (Invitrogen) according to the manufacturer's protocol.

To establish the impact of RNA oxidative damage on the ability to conduct mRNA profiling, 1 μg non-digested RNA was reverse-transcribed into cDNA using Protoscript® II and poly(dT) primers at 42° C. for 1 h followed by enzyme deactivation at 80° C. for 5 min. cDNA was diluted 5-20× before being amplified by qPCR with gene-specific primers (200 nM for all genes except 125 nM for MMP9), SsoAdvanced™ SYBR Green master mix, and a CFX Connect Real-Time System (BioRad). The PCR thermocycling protocol was 95° C. for 5 min and 40 cycles of 95° C. for 30 s, 50° C. for 30 s, and 72° C. for 1 min.

Anti-CD8 Enrichments of MOLT-3-Derived EVs

MOLT-3 cells were cultured at 37° C. and 5% CO₂ in RPMI-1640 with 10% FBS. FBS was depleted of background bovine EVs via ultracentrifugation (100,000 rcf, 18 h, 4° C.) with an L8-80M ultracentrifuge, Type 45 Ti rotor, 38 mm×102 mm (70 mL) polycarbonate tubes (Beckman Coulter), and a mechanical Harvard Trip balance (OHAUS). Tubes were sterilized with 10% hydrogen peroxide before use and disinfected with Virkon S when transferring between the centrifuge and culture hood. After ultracentrifugation, the FBS supernatant was decanted, mixed thoroughly to homogenize protein content, aliquoted, and stored at −20° C. Cells were transitioned into EV-depleted FBS for 1 week before obtaining MOLT-3 conditioned media by centrifugation (2000 rcf, 10 min).

EV microfluidic affinity-purification devices were modified with the PC linker and a monoclonal anti-CD8 Ab as described above. Before affinity-enrichment, EV microfluidic devices were washed with 400 μL blocking buffer (1% BSA, 1% PVP-40 in PBS) at 10 μL/min. Conditioned media (500 μL) was infused at 5 μL/min, then the device was washed with 400 μL 0.2% Tween 20 in TBS buffer and then 50 μL PBS at 10 μL/min. After LED exposure, released EVs were collected in 400 μL PBS (20 μL/min) and stored at −80° C. for subsequent analysis.

Nanoparticles Tracking Analysis (NTA)

Thawed samples were heavily vortexed, loaded into a 1 mL syringe (BD), and infused into the flow cell of a NanoSight LM10 NTA instrument (Malvern Panalytical) equipped with a 488 nm laser and NTA 2.3 software. In some cases, samples were diluted to ensure 10-100 EVs per frame. Imaging used a camera shutter setting of 1206, camera gain of 366, 90-160 s acquisition times, and 5 averaged replicates where the sample was advanced by ˜25 μL while the camera was off to image a random portion of the sample for each replicate. Processing used the Detection Threshold 20 and all other automatic settings. Between each sample, the flow cell was slowly flushed with 1 mL PBS then air four times, and flushing was verified by manually monitoring the number of nanoparticles observed in 300 μL PBS (˜0-1 per 100 μL). Nanoparticle concentrates were multiplied by each assay's elution volume to report the number of nanoparticles released.

Transmission Electron Microscopy (TEM)

Samples were heavily vortexed and spotted onto TEM grids. After 20 min, excess buffer was blot dried, and the grids were washed three times with a drop of nanopure water for 10 s. Grids were blot dried and stained with 5 μL 2% (w/v) uranyl acetate (0.22 μm filtered) for 10 s. Grids were blot dried, air dried for 15 min, and imaged with a Technai F20 XT Field Emission TEM (FEI).

LED-Induced mRNA Damage of EVs

A panel of genes (MMP9, PLBD1, FOS, CA4 and VCAN) was previously identified for diagnosing acute ischemic stroke (AIS) (M. G. Adamski, Y. Li, E. Wagner, C. Seales-Bailey, N. Bennett, H. Yu, M. Murphy, S. A. Soper, A. E. Baird, Med. Res. Arch. 2017, 5). A portion of this gene panel was used to determine if there was LED-induced damage of EV mRNA that may affect their expression profiles. MOLT-3 conditioned media was obtained from culture as described above. Cells were removed by centrifugation (300 rcf for 10 min), and EVs were precipitated using the ExtraPEG procedure (M. A. Rider, S. N. Hurwitz, D. G. Meckes, Jr., Sci. Rep. 2016, 6, 23978). To 3 mL conditioned media, we spiked an equal volume of PEG and NaCl (final concentration of 12% and 0.5 M, respectively), mixed by pipetting, incubated overnight at 4° C., centrifuged the sample (4000 rcf for 1 h), removed the supernatant, washed the pellet with ice cold PBS, resuspended the pellet in PBS (6 mL) by pipetting and vortexing, and aliquoted 1 mL portions into 35 mm diameter tissue culture dishes. Culture dishes were placed in an ice bath, inserted in the LED chamber, and either not exposed (control) or exposed for 2 min (18.5 J) by the LED. The EV suspension was then removed from the culture dish, and 1 mL of TRI Reagent was added to lyse EVs. The RNA was extracted using Direct-zol RNA extraction kit according to Zymo's guidelines and analyzed with a 2200 TapeStation and High Sensitivity RNA reagents (Agilent).

Reverse Transcription and Droplet Digital PCR (RT-ddPCR)

Complementary DNA (cDNA) was synthesized by performing RT with the Protoscript® II cDNA synthesis Kit and anchored d(T)23 VN primers according to New England Biolabs' protocol. cDNA product was used to generate droplets with the QX200 droplet generator, EvaGreen® Supermix, and gene specific primers (125 nM) followed by PCR amplification with the BioRad C1000 thermal cycler and the above thermocycling protocol. Final cooling was carried out at 4° C. Droplets were read with a BioRad QX200 droplet reader, and data analyzed using QuantaSoft™ software. All data were normalized to the total RNA concentration.

Photocleavable Heterobifunctional Linker Design

The photocleavable heterobifunctional linker strategy employs a 7-(diethylamino)coumaryl-4-methyl derivative that contains: (i) a central coumarin group that cleaves at the meta carbon, breaking the linker upon photoexposure, (ii) a primary amine with a short, 2-unit PEG spacer for EDC/NHS coupling to surfaces, and (iii) a COOH group for subsequent EDC/NHS activation and capture element immobilization. Rather than the more common o-nitrobenzyl PC group that cleaves upon UV-A exposure, which can damage cells and genetic material by reactive oxygen species (ROS) or photon absorption, the coumarin group cleaves via non-invasive blue light (400-450 nm). Further, the linker's good cleavage quantum efficiency (0.25) coupled with inexpensive, high power LEDs outputting light (2×10¹⁸ photons/s) enables rapid (<1 min) biomarker release without any labile or costly reagents, thereby enabling time-sensitive clinical applications and/or keeping the assay cost low. Additional benefits are achieved by simplifying the immobilization chemistry for the linker and affinity-selection antibody, which both use EDC/NHS coupling and does not require antibody modifications that incur antibody losses.

The reaction scheme for immobilizing the photocleavable heterobifunctional linker and an antibody as a capture entity (used as an example here) on a surface of a substrate is shown in FIG. 1B. However, it should be recognized that the antibody as the capture entity and the biomarker as the target substance to be captured can be varied as described herein, where the capture entity is configured to target the target substance for capture. As discussed above, the strategy employs two EDC/NHS reactions, the first immobilizing the linker to the surface and the second immobilizing the affinity-selection antibody to the linker. A potential caveat to this setup is that if the first EDC/NHS coupling is inefficient, then some free —COOH groups would remain on the surface, and during the second EDC/NHS reaction, some antibodies could attach to both the linker and directly to the surface, the latter being capable of affinity-selection but not for selective release of the target substance. To mitigate this potential side reaction, the reaction of the linker immobilization and EDC/NHS reactions is conducted in anhydrous solvent, thereby mitigating hydrolysis of the intermediate NHS ester and improving EDC/NHS conjugation efficiency, and to quench any unreacted NHS esters from the linker immobilization using a buffer containing primary amines (e.g., TRIS buffer).

FIG. 1B shows a design and reaction scheme of the photocleavable heterobifunctional linker. The linker's terminal amine attaches to COOH groups on UV/O₃-activated COC surfaces (used as an example here, but any COOH containing surface can be used) via EDC/NHS coupling. Any remaining, free NHS esters are quenched with TRIS buffer. The linker's carbonyl (e.g., COOH) group is then activated with EDC/NHS reagents for antibody coupling (or coupling to a capture entity having a reactive amine that reacts with the carbonyl, yielding a covalent linkage of the affinity-selection antibody to the surface through the linker. After affinity selection, isolated biomarkers (EVs or CTCs or others) are released by exposing the linker to blue light (400-450 nm), thereby cleaving the coumarin derivative at the meta carbon via a carbocation intermediate.

Monitoring Photolytic Products and Spectral Properties

The embodiments of the photocleavable heterobifunctional linker shown in FIG. 1C was analyzed for photolytic products after being exposed to light with a wavelength of about 400-450 nm while in water, which resulted in the illustrated products. Samples were taken at different time points (0, 1, 2, and 10 min). Photolysis products were monitored with UPLC (FIG. 1D) and identified by mass spectrometry. The intact PC linker (87%) concentration decreased with photo-irradiation time and at 10 min, the chromatographic peak for the intact linker completely disappeared. Major photolysis product (larger product) was present in small amounts (5%) for the initial sample and that amount increased with the photo-irradiation up to 79%. In addition, UV-visible absorption (FIG. 1E) and fluorescence measurements (FIG. 1F) were used to monitor the photocleavage reaction. From the spectra shown in FIG. 1B, very little changes in the absorption spectra were seen as a function of irradiation time. However, there was a slight decrease in the fluorescence from the coumarin as a function of photocleavage of the starting material (FIG. 1C).

FIG. 1D shows ultra-high performance liquid chromatography (UPLC) of the photocleavage of the linker using 400-450 nm light for exposure times of 0, 1, 2, and 10 min. The chromatography used a C18 column with a aqueous buffer and acetonitrile as the mobile phase. FIG. 1E shows the UV/vis spectra of the intact photolinker as a function of exposure time. FIG. 1F shows the fluorescence emission spectra of the photo-irradiated linker as a function of time.

Stability of UV/O₃-Actived COC in Anhydrous Solvents

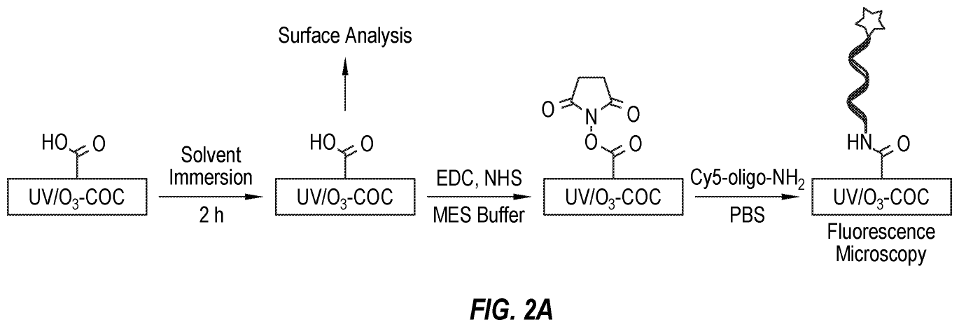

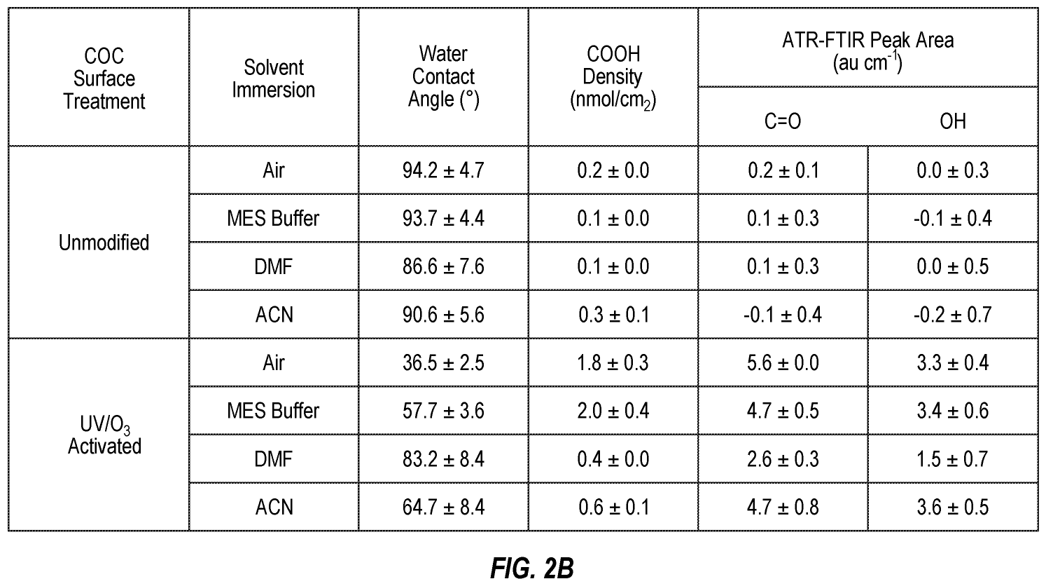

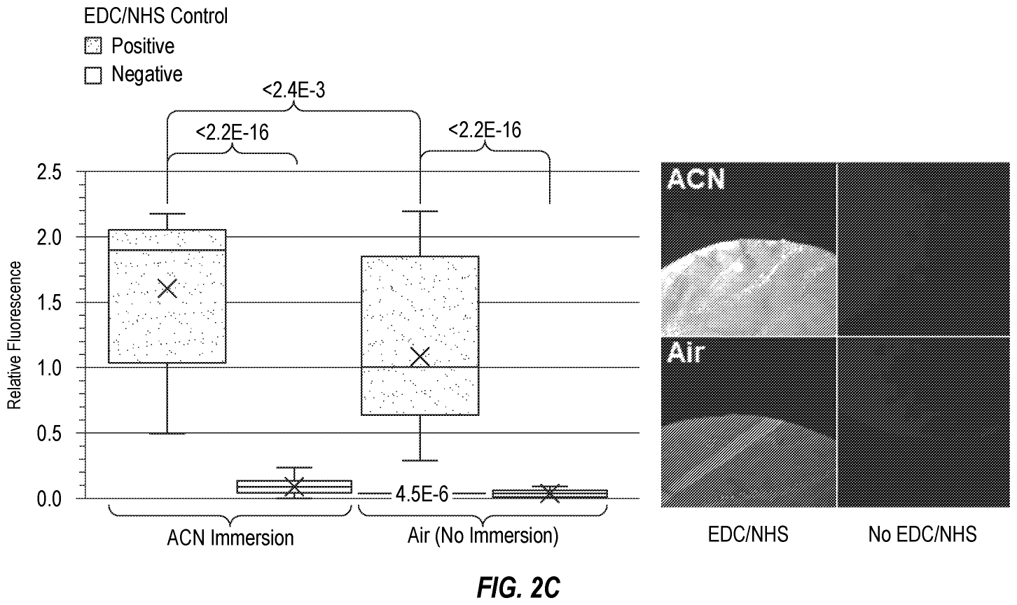

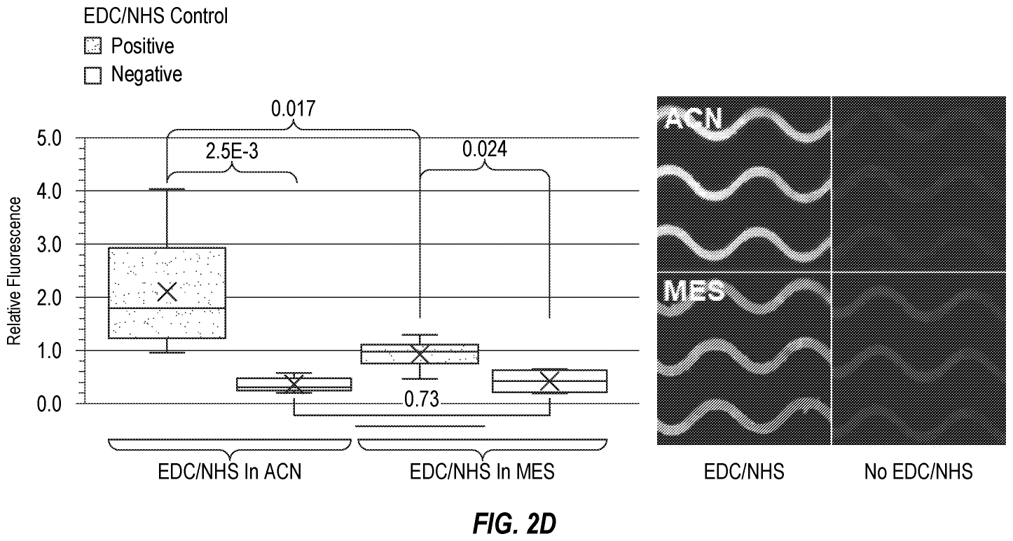

COC is well-known for exceptional solvent resistance, but the effect of anhydrous solvents on the stability of —COOH groups for UV/O₃-activated COC has not been investigated. Planar COC surfaces were tested, either unmodified or UV/O₃-activated, left in air or immersed in buffer (MES, pH 4.8) or anhydrous solvents—acetonitrile (ACN) or dimethylformamide (DMF)—for 2 h (FIG. 2A). The surfaces were rinsed with water, dried, then evaluated via water contact angles (WCAs), —COOH densities via a colorimetric TBO assay, or ATR-FTIR (FIG. 2B) including water contact angles (WCAs), —COOH densities via a colorimetric TBO assay, and ATR-FTIR spectroscopy. Based on these data, one solvent was selected for testing bio-molecule coupling by EDC/NHS-activation then spotting of 3′-NH2, 5′-Cy5 ssDNA oligonucleotide fluorescent reporters (FIG. 2C). These reporters are a surrogate for antibody immobilization efficiency used because fluorescent antibodies are labeled via EDC/NHS coupling, rendering surface immobilization infeasible.

FIG. 2C shows ACN pre-treatment of UV/O3-COC yielded higher loads of Cy5-oligonucleotides compared to MES (N=33-35). FIG. 2D shows immobilization in solvent provides further gains that were observed in fluorescence microscopy by EDC/NHS coupling in ACN (N=4).

The UV/O₃-activated COC surfaces that were not exposed to buffer or solvent, as expected, exhibited increased wettability (36.5±2.5° WCA) and —COOH surfaces (density=1.8±0.3 nmol/cm²; determined by TBO assay) was supported by the appearance of carbonyl and hydroxyl peaks in ATR-FTIR spectra. Treatment with MES buffer increased surface hydrophobicity (57.7±3.6° WCA) and slightly decreased —COOH surface densities (1.2±0.4 nmol/cm²) without an appreciable change in the ATR-FTIR spectra, which generally probes deeper (0.5-5 μm) into the sample. Based on previous studies, UV/O₃ surface activation produces a heterogenous spectrum of oxidized functionalities and, to a degree dependent on the polymer itself, can cause scissioning of the polymer chains and fragmentation of the surface. While COC appeared to be more resistant to fragmentation than, for example PMMA, fragmentation is likely to occur to some extent. We suspect that MES buffer immersion resulting in decreased wettability and —COOH densities yet without significantly altering ATR-FTIR measurements of the “bulk” surface is likely caused by solubilization of carboxylated polymer fragments.

DMF treatment increased the WCA appreciably (83.2±8.4°) and reduced —COOH densities to 0.4±0.0 nmol/cm², near the nonspecific limit of the TBO assay. Further, ATR-FTIR peak areas were reduced after DMF treatment. Along with the altered WCAs in the unmodified COC control, these data indicated solvent penetration and partial solubilization of the surface even though no degradation or swelling of the bulk material was observed—only harsh solvents such as dichloromethane produced these artifacts for COC.

ACN treatment yielded a surface comparable to the MES buffer treatment (FIG. 2B). The only disparity between ACN and MES treatment was a 68% decrease in —COOH densities (0.6±0.1 nmol/cm²), although the consequences for biomolecule immobilization are limited by the stoichiometric ratio of smaller —COOH groups and larger biomolecules, such as antibodies. For example, a theoretical monolayer of —COOH groups is 0.83 nmol/cm², whereas a monolayer of larger oligonucleotides or antibodies is 13.2 μmol/cm² or 0.85 μmol/cm², respectively.

The ability to immobilize Cy5-oligonucleotide fluorescent reporters by EDC/NHS coupling (in MES buffer) to UV/O₃-activated COC surfaces that were left in air or immersed in ACN for 2 h was tested. A significant improvement in Cy5-oligonucleotide loads were achieved by immersing the UV/O₃-activated COC surfaces in ACN prior to immobilization (FIG. 2C), which is evidenced by an 89% increase in fluorescence signal (p=0.0024). It is noted that after ACN immersion, washing, and drying, all incubation/washing buffers were identical for the two substrates including imaging conditions. The beneficial effects of ACN immersion were extended to the microfluidic device by performing the EDC/NHS reaction for Cy5-oligonucleotide immobilization in either MES buffer or anhydrous ACN (FIG. 2D). It was observed that a similar improvement, a 77% increase in median fluorescence (p=0.017), by exchanging the aqueous MES system with anhydrous ACN. While the TBO assay showed decreasing COOH densities, all values are in excess for oligonucleotides (63 COOH groups per oligonucleotide if both are in a monolayer), and similar improvements were observed by fluorescence microscopy for ACN surface treatment regardless of whether the EDC/NHS reaction was conducted in MES buffer or ACN (FIGS. 2C, D). Thus, it is possible that ACN is reorganizing the UV/O₃-actived COC surfaces and/or removing fragmented polymer and providing improved sterics for biomolecules to react with surface-confined COOH groups.

Also shown in FIGS. 2B-2D are stability data of UV/O₃-activated COC surfaces exposed to anhydrous solvents. FIG. 2B shows planar COC surfaces were UV/O₃-activated and left in air or submersed in MES buffer or anhydrous solvents DMF (dimethylformamide) and acetonitrile (ACN) for 2 h. Substrates were washed with water and dried for surface analyses—water contact angle, TBO assay for COOH densities, and ATR-FTIR analysis for functional groups containing carbonyl and hydroxyl moieties. To evaluate surface changes that would affect antibody immobilization, surfaces were EDC/NHS-activated before spotting a 5′-Cy5, 3′-NH₂ single stranded DNA oligonucleotide reporter, and fluorescence microscopy was used to evaluate the efficiency of biomolecule conjugation. FIG. 2B shows surface analyses of unmodified and UV/O₃-activated, all performed in triplicate, indicated that surface treatment with MES buffer and anhydrous ACN yielded similar surface properties; thus, further testing was focused on ACN. FIG. 2C shows ACN treatment of the UV/O₃-activated COC surface prior to EDC/NHS coupling improved biomolecule conjugation efficiency relative to aqueous MES buffer. Each data set included 35 Cy5-oligo spots imaged on 6 replicates. FIG. 2D shows findings from FIG. 2C extend to on-chip immobilization, where Cy5-oligo loads increased when the EDC/NHS ester formation was conducted in anhydrous ACN versus buffer while keeping all other conditions constant. Data shown includes 4-7 replicates for each condition. For FIGS. 2C and 2D, the box plots show the average (X-mark), median (mid-line), upper and lower quartiles (box edges), and range (error bars). Given the non-Gaussian profile of these data, Wilcoxon Rank Tests were performed for statistical comparison (p-values shown).

FIGS. 2C-2D show the 5′-NH2-, 3′-Cy5-oligonucleotide direct attachment to UV/O₃—COC surfaces. FIG. 2C shows the ACN pre-treatment of UV/O3-COC yielded higher loads of Cy5-oligonucleotides compared to MES (N=33-35). FIG. 2D shows further gains were observed in fluorescence microscopy by EDC/NHS coupling in ACN (N=4).

Photocleavable Heterobifunctional Linker Immobilization and Cleavage

The linker was immobilized in UV/O₃-activated COC devices using ACN for the EDC/NHS reaction and ACN and triethylamine (TEA) organic base for linker conjugation to the formed NHS ester. Three concentrations of linker were tested for the immobilization reaction—2.65 mM, 0.530 mM, and 0.106 mM—corresponding to reaction excess of 5×, 1×, and 0.2× relative to a theoretical monolayer of linker (0.51 nmol/cm², 1.84×1015 molecules per device). After linker immobilization, the device was protected from ambient light by wrapping the device in Rubylith® film, which absorbs light throughout the linker's absorption spectrum (200-450 nm, λₘₐₓ=385 nm).

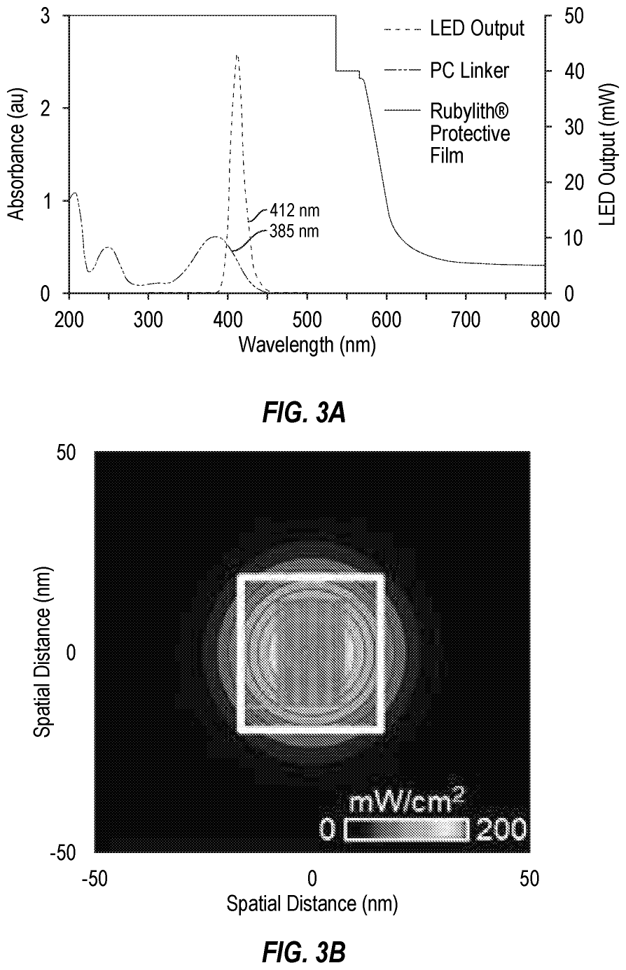

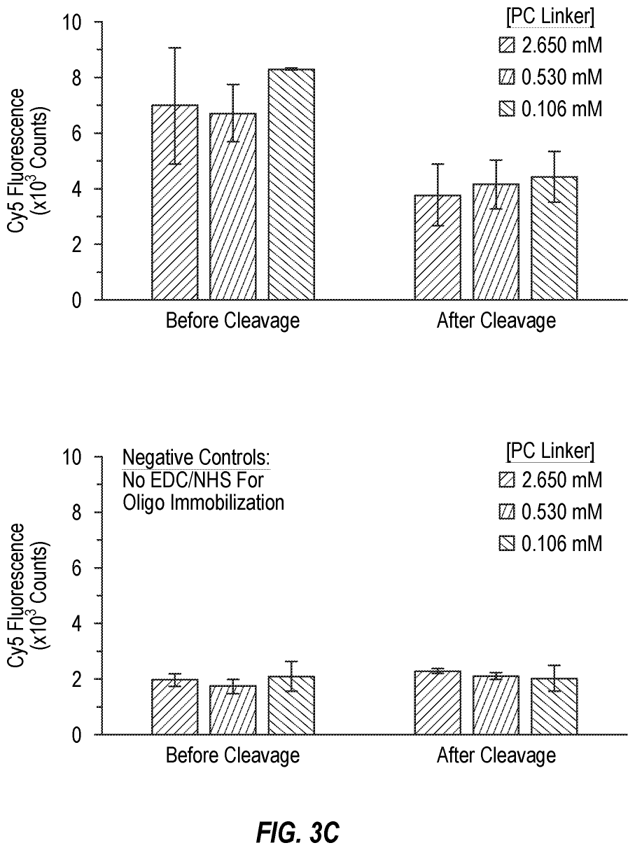

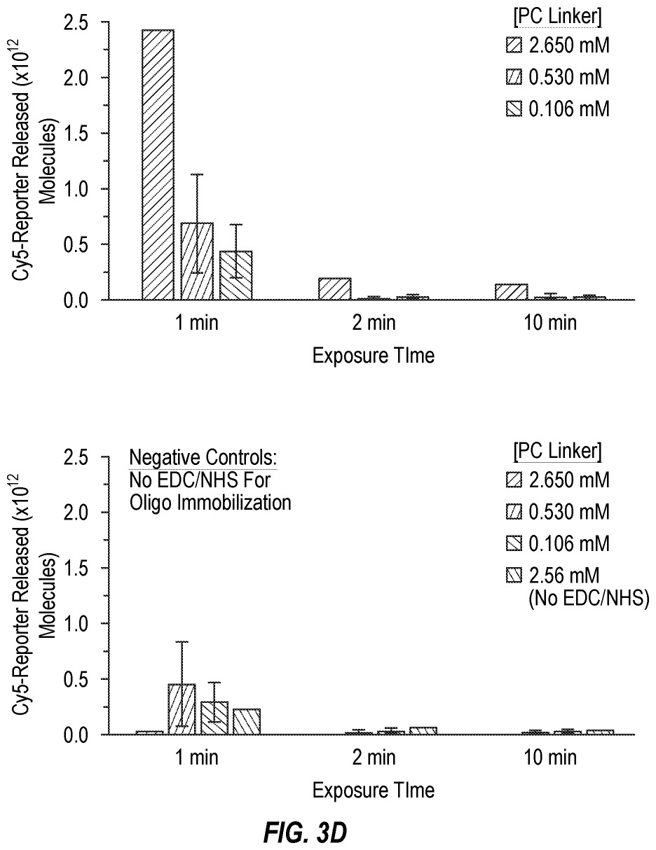

For linker cleavage, a photoexposure chamber was constructed using an LED outputting 885 mW light ranging from 385-470 nm (λₘₐₓ=412 nm), which overlaps significantly with the linker's absorption spectrum (FIG. 3A) and does not expose biological samples to intense UV-A radiation. To illuminate devices, the LED was placed 24 mm from the device surface to allow the LED's innate divergence (60°) to provide a spot diameter of 90 mm, which provided relatively homogenous illumination over the device's surface area (182±22 mW/cm²; FIG. 3B). These measurements were not performed inside the polished aluminum housing of the photoexposure chamber, which likely improves the illumination uniformity via internal reflections. Lastly, we note that larger or smaller devices can be accommodated without any additional optical elements by simply changing the distance between the LED light source and the device surface.

FIG. 3A shows the LED's spectral output, the absorbance spectra of the PC linker (measured at 526 μM in PBS, pH 7.4), and the Rubylith® film used to protect devices from ambient light and premature photocleavage. FIG. 3B shows that for photoexposure, the Rubylith® film is removed, and devices were inserted into an aluminum exposure chamber, where the LED was centered with a 90 mm spot size over the device. The LED's spatial flux was measured by rastering a sensor underneath the LED and fitting a 2-dimensional Gaussian (R2=0.9986), which showed a uniform exposure (34±4 mW/cm2) over the device.

The PC linker was linked to UV/O₃-oxidized COC devices using ACN for the EDC/NHS reaction and ACN and triethylamine (TEA) organic base for PC linker conjugation to the formed NHS ester. Three concentrations of PC linker were tested for the immobilization reaction—2.65 mM, 0.530 mM, and 0.106 mM—corresponding to reaction excesses of 5×, 1×, and 0.2× relative to a theoretical monolayer of PC linker (0.56 nmol/cm2, 1.82×1015 molecules per device). Before PC linker immobilization, the device was protected from ambient light by wrapping the device in Rubylith® film, which absorbs light throughout the PC linker's absorption spectrum (FIG. 3A). Following selection of either CTCs or EVs, the film was removed before LED exposure.

After immobilization, the linker was not visible by fluorescence microscopy using a DAPI filter set, most likely due to high autofluorescence of the UV/O₃-activated polymer at these wavelengths. Thus, we continued the immobilization scheme (FIG. 1B) by activating the linker's —COOH group with EDC/NHS and immobilized Cy5-oligonucleotide reporter to the linker. Fluorescence microscopy was used to visualize the amount of Cy5-oligonucleotide immobilized to the linker (FIG. 3C). The Cy5-oligonucleotide signal was 3.5-4× higher in positive controls versus nonspecific controls, where the EDC/NHS coupling reagents were not included during the Cy5-oligonucleotide immobilization. To confirm that the Cy5-oligonucleotides were indeed coupled through the linker, we cleaved the linkers using the LED photoexposure system and washed released Cy5-oligonucleotide reporters into the effluent, where fluorometry could be used to quantitate the number of Cy5-oligonucleotide reports within the devices absent of any interfering autofluorescence from the UV/O₃-activated device.

In all positive controls, higher levels of Cy5-reporter were eluted compared to nonspecific controls, and there appears to be a dependence on the concentration of linker used in the immobilization reaction and the amount of biomolecules immobilized within the device (FIG. 3D). This is somewhat surprising, because while the lowest concentration represents a 0.2× reaction excess to a monolayer of linker molecules, it represents a reaction excess of ˜8× relative to a monolayer of the larger Cy5-oligonucleotide reporter. Thus, while not saturating the surface with linker, excess linker should be available for the biomolecule immobilization.

FIG. 3A shows the LED's spectral output, and the absorbance spectra of the linker (measured at 526 mM in PBS, pH 7.4) and the Rubylith® film used to protect devices from ambient light and premature photocleavage. FIG. 3B shows that for photoexposure, devices are inserted into an aluminum exposure chamber, where the LED is centered with a 90 mm spot size over the device (shown here as the sinusoidal CTC device—26 mm×16 mm). The LED's spatial flux was measured by rastering a sensor underneath the LED and fitting a 2-dimensional Gaussian (R²=0.9986), which shows a relatively uniform exposure (182±22 mW/cm²) over the device. FIG. 3C shows the linker was immobilized at three concentrations (reaction excesses of 5×, 1×, and 0.2×) and labeled with Cy5-oligonucleotide fluorescent reporter by EDC/NHS conjugation, then the device was exposed beneath the LED for 10 min to cleave the linker and release the Cy5-oligonucleotide. Fluorescence microscopy results are shown before and after linker cleavage for positive and negative controls. FIG. 3D shows the amount of Cy5-oligonucleotide released after different exposure times was collected in the effluent quantified by fluorometry. In FIGS. 3C and 3D, error bars show the range of data obtained from duplicate measurements if available.

Catch and Release of Circulating Tumor Cells (CTCs) Using the Linker

To evaluate the ability of the photocleavable heterobifunctional linker to catch and release CTCs seeded into whole blood, we immobilized a monoclonal antibody directed against membrane antigens of two different types of CTCs, those of an epithelial origin (MCF7) and those of a mesenchymal origin (Hs578T). EpCAM expressing CTCs represent the epithelial type and those that express fibroblast activation protein α, FAPα, are the mesenchymal type. FAPα is a cell surface protease that plays a role in facilitating cell invasion into the extracellular matrix (ECM) and is expressed in >90% of human epithelial cancers. This protein is differentially expressed within cell membrane protrusions (invadopodia) and can degrade a variety of substrates. The CTC selection microfluidic devices used for these studies were made from cyclic olefin copolymer (COC) via hot embossing. Whole blood enters the CTC selection device through a single inlet channel, passes through a parallel array of narrow sinusoidally-shaped CTC selection channels and exits through a single outlet channel. The sinusoidal channels were covalently decorated with a particular monoclonal antibody (mAb) type following UV/O₃ activation of the polymer. Sinusoidally-shaped microchannels for the positive selection of CTCs provided high recoveries and exquisite purity from whole blood.

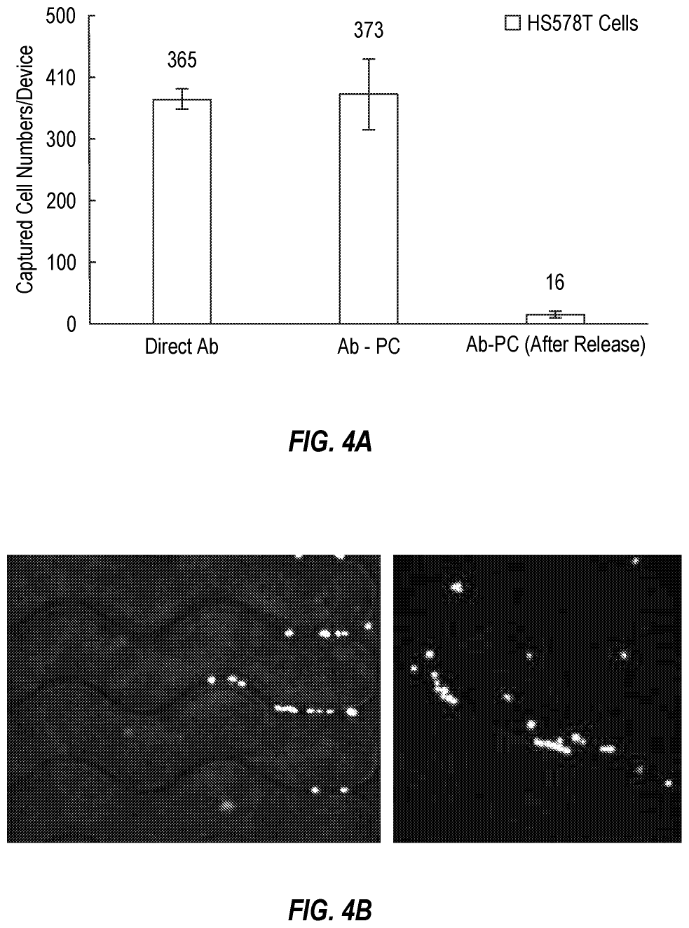

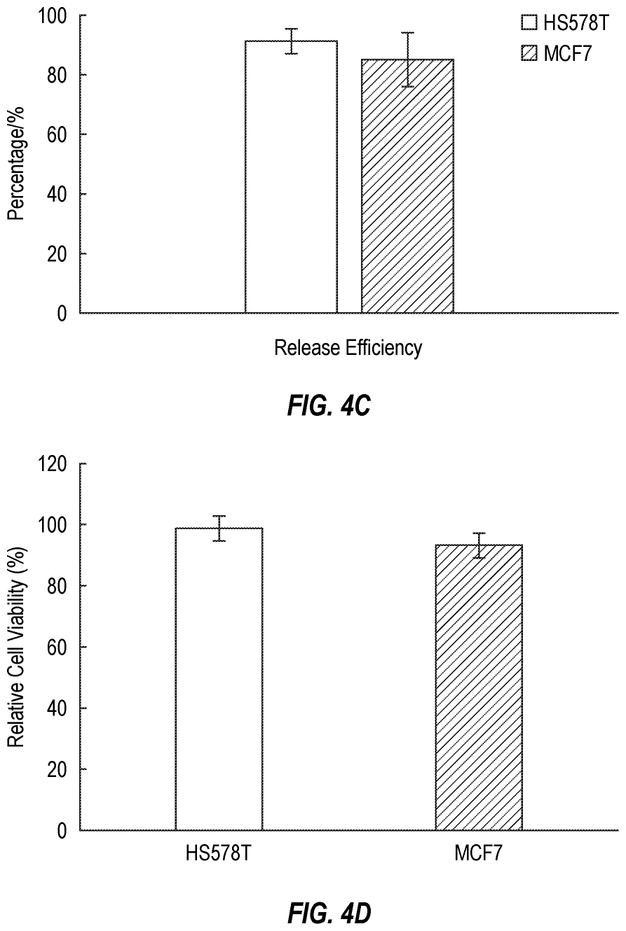

In the first set of experiments, the selection antibody was either attached directly to the activated polymer surface using EDC/NHS coupling chemistry or through the photocleavable bifunctional linker. The results indicated that whether using direct attachment or through the linker, the Hs578T CTCs showed similar recoveries (FIG. 4A). We have shown that the recovery of this cell line using anti-FAPα antibodies is >78%. Fluorescence microscope images of captured and released CTCs stained with a nuclear dye, DAPI, is shown in FIG. 4B indicating the intact nature of the cells following processing use the CTC assay. As can be seen from FIG. 4C, the release efficiency for both CTC types was >90% using a 2 min blue light exposure. This is better than the ssDNA enzymatic cleavage assay we reported, which showed ˜85% release efficiency following a 30 min reaction. Finally, we evaluated the viability of the CTC following capture and release and the data is shown in FIG. 4D. From the data, following running the CTCs through the complete assay, near 100% of the CTCs remain viable.

Breast Cancer Circulating Tumor Cells (CTCs)

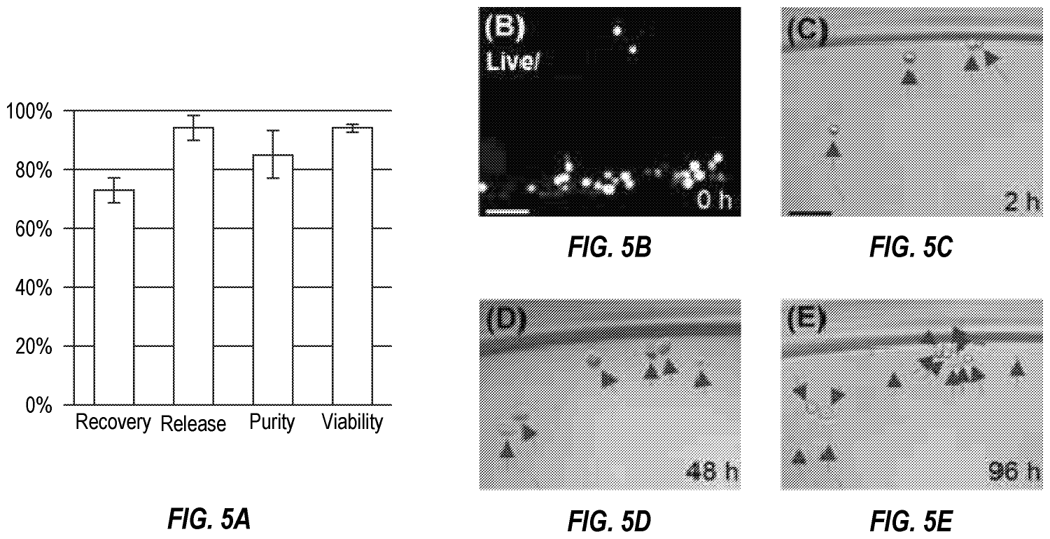

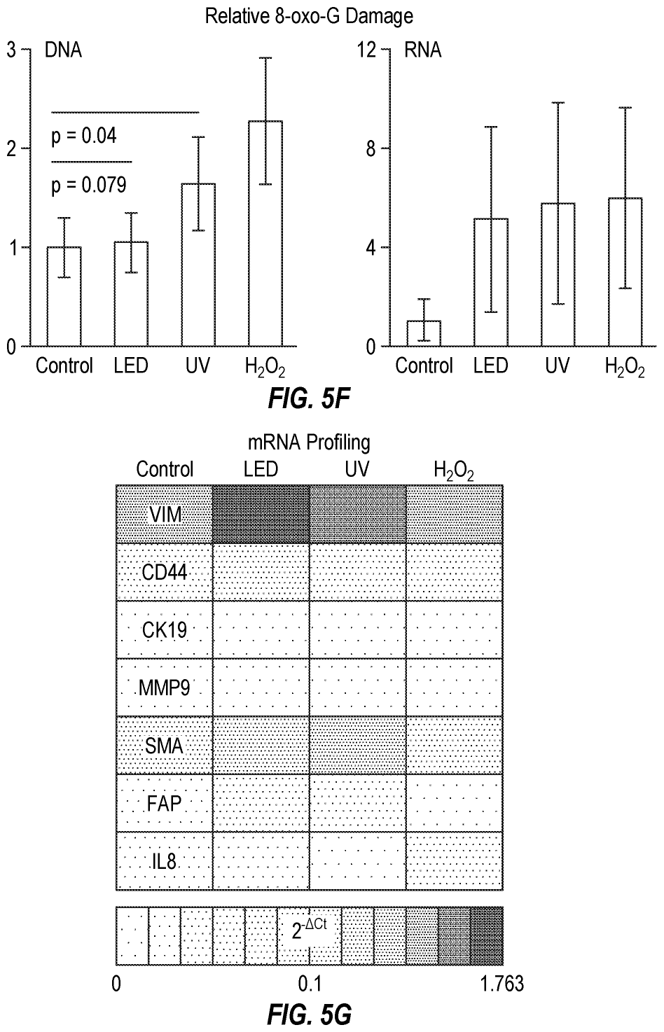

The PC linker was used to immobilize anti-EpCAM Abs in a sinusoidal microfluidic device for CTC affinity-enrichment. SKBR3 cells (metastatic breast cancer) spiked into healthy blood (69-269 SKBR3 cells/mL) were used, and pre-stained with Hoechst dye and, after enrichment, stained all cells with SYTO 82, another membrane-permeable, nuclear dye that is spectrally distinct from Hoechst. SKBR3 cells were dual-stained, while leukocytes were only stained with SYTO 82, enabling analysis of recovery by self-referencing and purity (FIG. 5A). FIG. 5A shows the performance of sinusoidal microfluidic device using PC linker for anti-EpCAM enrichment of SKBR3 cells spiked into whole blood (N=3).

SKBR3 cells were enriched with 85±8% purity (16-38 leukocytes/mL) and 73±4% recovery (47-202 cells), slightly lower than found for the dU linker (85±4%) and direct Ab attachment (96±12%). The PC linker was used to immobilize IgG 2A isotype Ab to evaluate nonspecific cell recovery (3±2%). The release of SKBR3 cells was rapid with 94±4% efficiency after 2 min of LED exposure. Two other breast cancer cell lines were also enriched and released with 88±10% and 91±4% efficiency for EpCAM(+) MCF7 and FAPα(+) Hs578T cells, respectively.

After exposure to the visible LED light, released SKBR3 cells had 94±1% viability, same as controls (FIG. 5B), and could be propagated in culture for 96 h (FIGS. 5C-5E). Similarly, exposed MCF7 and Hs578T cells had 96±6% and 99±3% relative cell viability, respectively. However, UV irradiation can damage nucleic acids through photo-absorption and indirect oxidation (8-oxoguanines, 8-oxo-G). FIG. 5B shows that LED release had no effect on viability, and FIGS. 5C-5E show the released cells in culture for 2-96 h (Scale bars=100 μm).

The 8-oxo-G levels were measured in RNA and DNA for Hs578T cells exposed to visible LED versus UV light (both 18.5 J) and compared both to H₂O₂, which is known to generate oxidative damage in DNA and RNA via oxygen free radicals. DNA damage was not detected for LED exposure but was present with UV irradiation (FIG. 5F). Both exposures generated 8-oxo-G damage in RNA at comparable levels to that of H₂O₂(FIG. 5F). Single-stranded RNA is easily oxidized and can protect genomic DNA from damage and subsequent mutations through imperfect repair pathways. A limited gene panel consists of mesenchymal and epithelial to mesenchymal transition (EMT) markers were selected to determine the impact of mRNA oxidative damage to their expression. However, no treatment altered these gene's mRNA expression compared to control cells (FIG. 5G). Thus, visible LED exposure did not affect mRNA expression analysis or cause DNA damage, whereas UV irradiation induced DNA 8-oxo-G damage. Such DNA damage could cause false positives for clinical single nucleotide polymorphism analysis, especially at the single cell level common to CTCs.

Molt-3 Cell Line Extracellular Vesicles (EV)

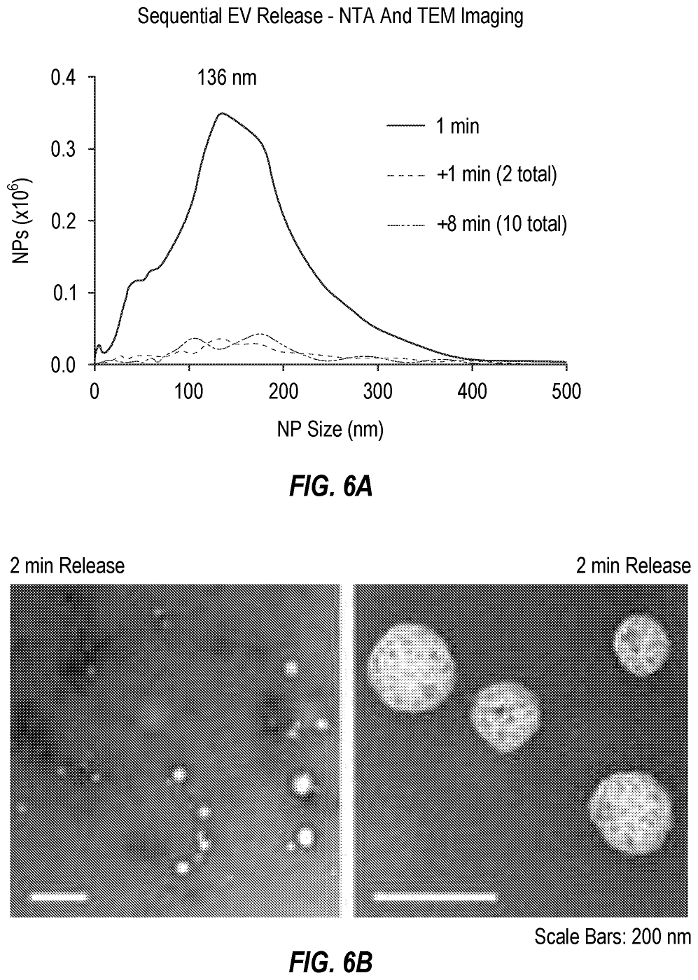

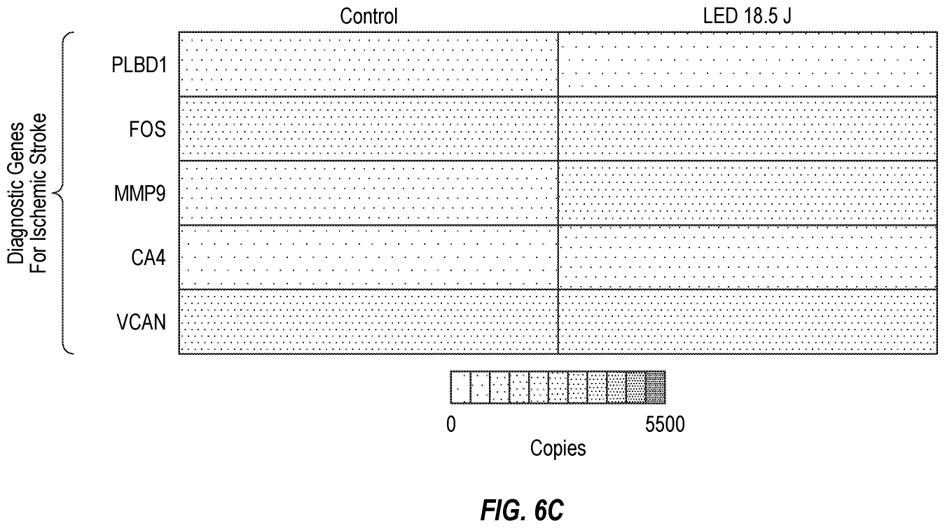

The PC linker and anti-CD8 Abs was immobilized in a UV/O₃—COC microfluidic device specially-designed to enrich EVs. The expression of CD8 antigen in Molt3 cells was reported as 13.5%. Therefore, we processed culture media conditioned by MOLT-3 cells to affinity select CD8(+) EVs. Affinity selected CD8(+) EVs were photo released for NTA and TEM imaging. From 500 μL media, we enriched 8.2±0.2×10⁷ nanoparticles (NPs) with an EV size of ˜136 nm, similar to TEM imaging (FIGS. 6A and 6B). EV release was rapid with 82±6% of NPs being released after 1 min LED exposure, and 91±5% were released in 2 min. Further, the expression of stressed gene panel was tested by droplet digital PCR after 2 min of LED exposure and compared with control EVs. LED exposure (2 min) did not affect EV-mRNA expression profiling (FIG. 6C). Thus, the PC linker strategy is well suited to reduce the acute ischemic stroke (AIS) assay workflow by >58 min compared to an enzymatic release strategy.

Antigen Expression Correlated to Release Efficiency

For the affinity-enriched cell lines, antigen expression was analyzed versus isotype controls by flow cytometry. MCF7 had the highest expression of EpCAM (125× IgG) followed by lower expression of EpCAM and FAPα in the SKBR3 and Hs578T cell lines (20× and 6× IgG, respectively). For enrichment, anti-EpCAM antibodies were used for enriching MCF7 and SKBR3 cell lines, while anti-FAPα antibodies were used for enriching the Hs578T cells. Release efficiencies were 88±10%, 94±4%, and 91±4% for the MCF7, SKBR3, and Hs578T cell lines, respectively. It was observed that a negative correlation (r=−0.81) between antigen expression and release efficiency. The rate of release depends on the number of Ab-antigen interactions. When the surface antigen expression is high, more interactions can form, and more time is required to cleave the bonds and release captured biomarkers.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

1. A photocleavable heterobifunctional linker comprising:
a structure of Formula A, 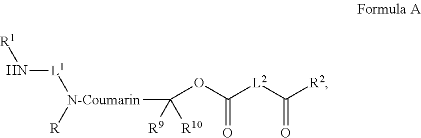
wherein:
coumarin is any coumarin or coumarin derivative;
R, R⁹, and R¹⁰ are each independently a chemical moiety;
R¹ is a hydrogen, protecting group, leaving group, substrate, or capture entity;
R² is a hydrogen, hydroxyl, halide, alkoxy, anhydride, amino, protecting group, leaving group, substrate, or capture entity;
L¹ is a sub-linker; and
L² is a sub-linker.

2. The linker of claim 1, comprising a structure of Formula B, 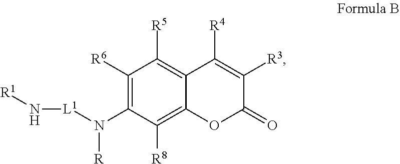
wherein:
one of R³⁻⁶ or R⁸ is a second linker arm having a structure of: 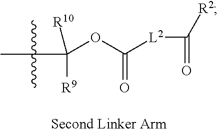
wherein the others of R³⁻⁶ or R⁸ are as defined for R.

3. The linker of claim 1, comprising a structure of Formula C, 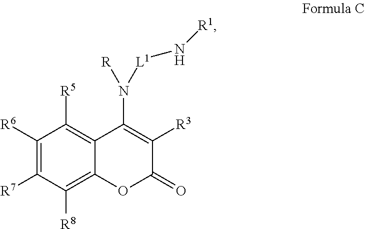
wherein:
one of R³ or R⁵⁻⁸ is a second linker arm having a structure of: 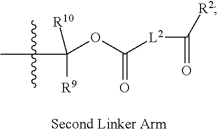
wherein the others of R³ or R⁵⁻⁸ are as defined for R.

4. The linker of claim 1, comprising a structure of Formula D, 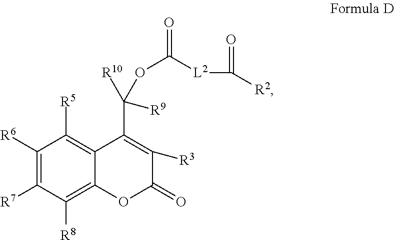
wherein:
one of R³ or R⁵⁻⁸ is a first linker arm having a structure of: 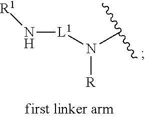
wherein the others of R³ or R⁵⁻⁸ are as defined for R.

5. The linker of claim 1, comprising a structure of Formula E, 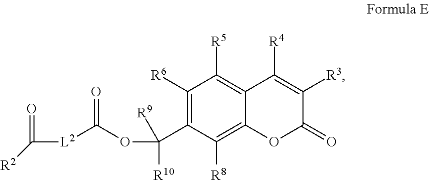
wherein:
one of R³⁻⁶ or R⁸ is a first linker arm having a structure of: 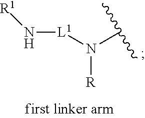
wherein the others of R³⁻⁶ or R⁸ are as defined for R.

6. The linker of claim 1, comprising a structure of Formula F, 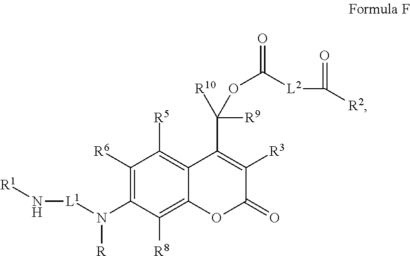
wherein:
R³ or R⁵⁻⁶ or R⁸ are as defined for R.

7. The linker of claim 1, comprising a structure of Formula G, 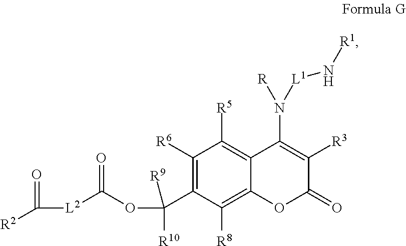
wherein:
R3 or R⁵⁻⁶ or R⁸ are as defined for R.

8. The linker of claim 1, comprising a structure of Formula H, Formula I, Formula J, Formula K, Formula L, Formula M, Formula N, Formula O, Formula P, Formula Q, or Formula R

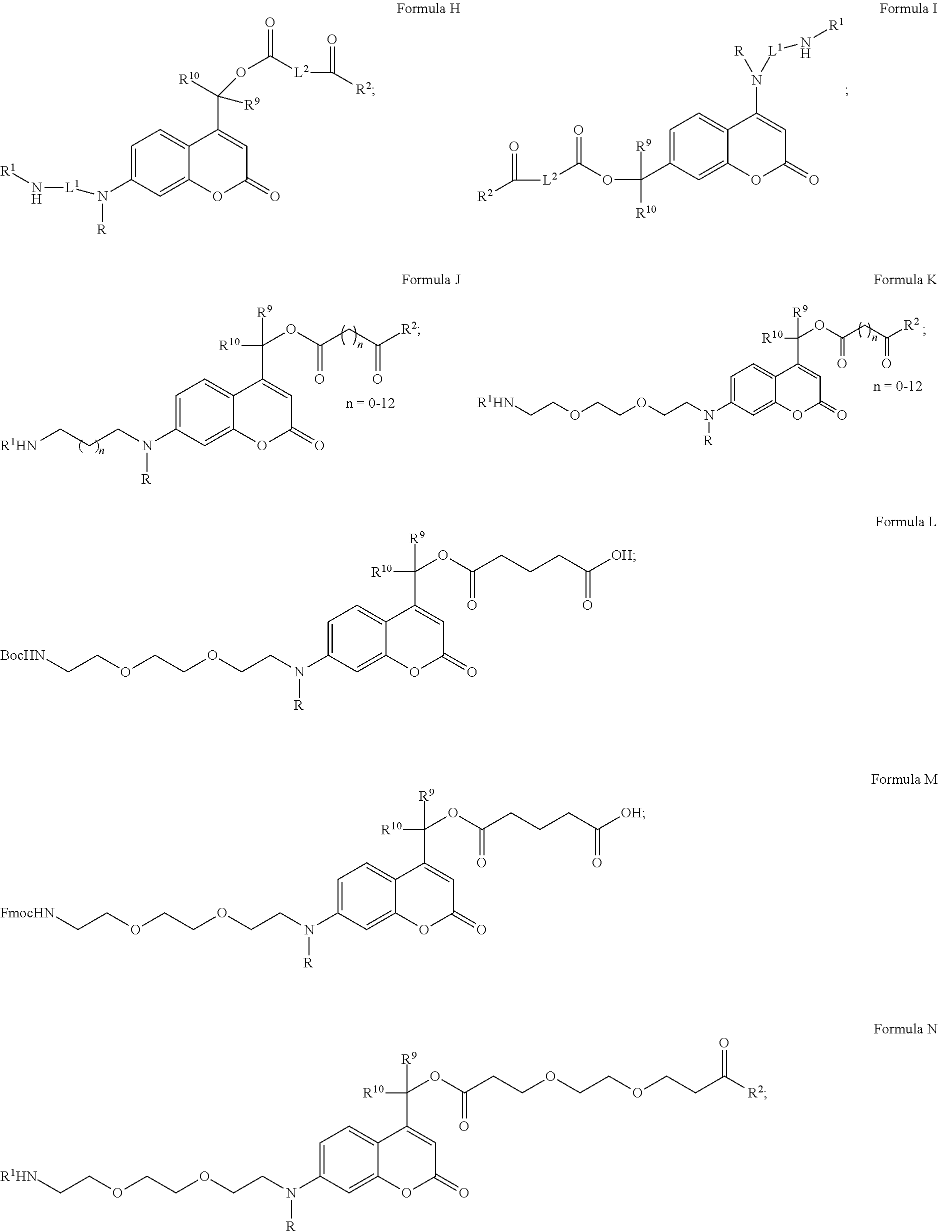

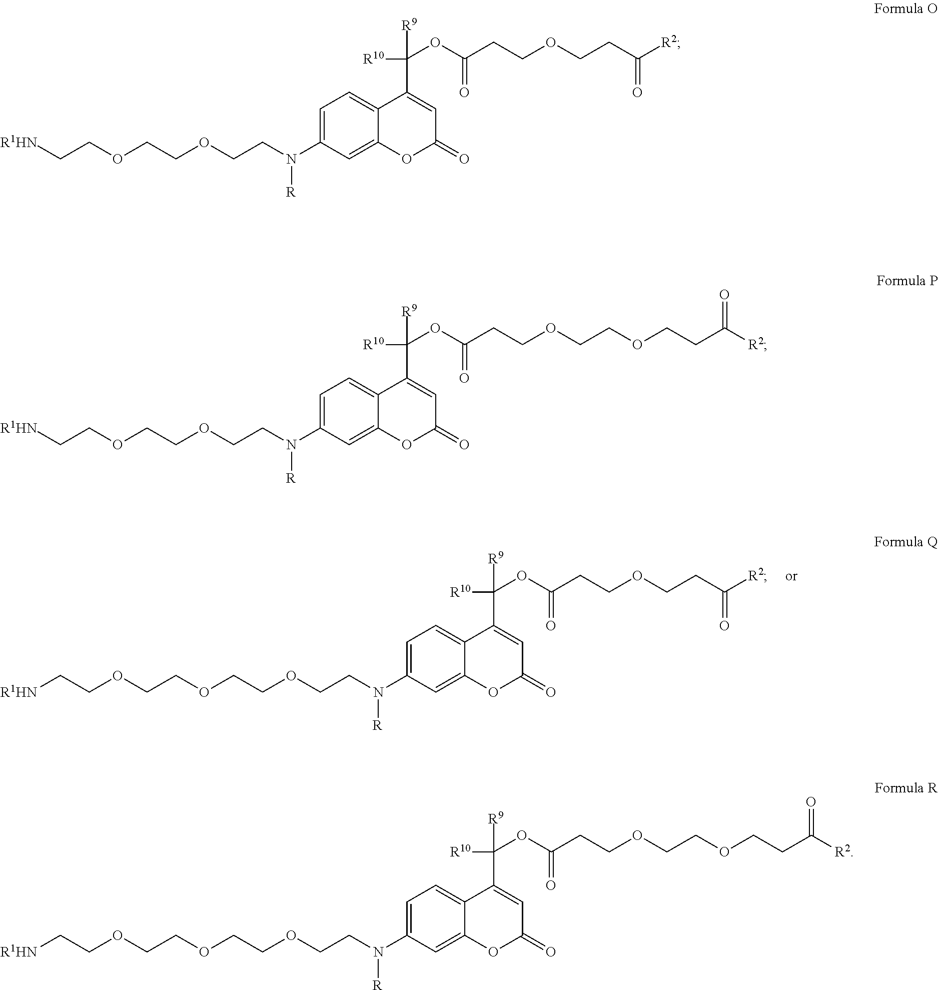

9.-21. (canceled)

22. The linker of claim 1, wherein R, R⁹, and R¹⁰ are each independently selected from hydrogen, methyl, ethyl, propyl, butyl, cyclopropyl, cyclobutyl, cyclohexyl, or combinations thereof.

23. The linker of claim 1, wherein R¹ is selected from hydrogen, tert-butyloxycarbonyl (Boc), 9-a fluorenylmethyloxycarbonyl (Fmoc), trifluoroacetyl, benzyl, 2-nitrophenylethyl carbamate or 6-nitroveratryl carbamate, fluoride, trimethylsilylethyloxycarbonyl (Teoc), or 1,3-dithian-2-ylmethoxycarbonyl (Dmoc).

24. The linker of claim 1, wherein R² is selected from hydrogen, hydroxyl, halide, alkoxy, anhydride, amine, or a carboxyl protecting group selected from an alkyl ester, aryl ester, tert-butyl ester, ester of 2,6-disubstituted phenol, silyl esters, or oxazoline.

25.-26. (canceled)

27. The linker of claim 1, wherein the L¹ sub-linker and L² sub-linker each independently includes alkyls, ethylene glycols, propylene glycols, ethers, esters, amides, oligoethylene glycols, polyethylene glycols, polypropylene glycols, or linker derived from amino-PEG-amine, or combinations thereof.

28. (canceled)

29. The linker of claim 1, wherein the capture entity is selected from the group of antibody, aptamer, peptide, protein, ligand, or receptor.

30. The linker of claim 1, wherein the substrate is selected from the group of well bottom, particle, bead, magnetic bead, porous member, non-porous member, solid member, microchannel, microchamber, reservoir, or combination thereof.

31. The linker of claim 1, wherein one of R¹ or R² is the substrate and the other of R¹ or R² is the capture entity.

32. A method of synthesizing the linker of claim 1, comprising:
providing a coumarin having the following structure, 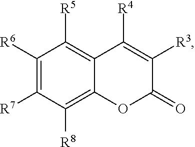
wherein one of R³-R⁸ is a leaving group and another of R³-R⁸ is a protecting group and the rest of R³-R⁸ are each individually a chemical moiety;
reacting the leaving group with a precursor of a first linker arm such that the first linker arm replaces the leaving group, 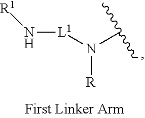
wherein R is a chemical moiety and R¹ is an amine protecting group;
converting the protecting group to an alcohol group; and
reacting the alcohol group with a precursor of a second linker arm so as to form an ester with the oxygen of the alcohol group in the second linker arm, 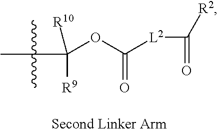
wherein R² is a carbonyl protecting group; and
R⁹ and R¹⁰ are each individually a chemical moiety.

33. The method of claim 32, further comprising:
deprotecting the amine protecting group to provide a primary amine; and
coupling the primary amine of the first linker arm with the substrate.

34.-35. (canceled)

36. The method of claim 32, further comprising:
deprotecting the carbonyl protecting group to provide a carboxylic acid; and
coupling the carboxylic acid to the capture entity.

37.-40. (canceled)

41. A capture device comprising:
the photocleavable bifunctional linker of claim 1 having a structure of Formula A, 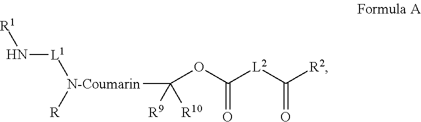
wherein:
coumarin is any coumarin or coumarin derivative;
R, R⁹, and R¹⁰ are each independently a chemical moiety;
R¹ is a substrate;
R² is a hydrogen, hydroxyl, halide, alkoxy, anhydride, amino, protecting group, leaving group, or capture entity;
L¹ is a sub-linker; and
L² is a sub-linker.

42. The capture device of claim 41, wherein the capture entity is selected from the group of antibody, aptamer, polypeptide, protein, ligand, or receptor.

43. The capture device of claim 41, wherein the substrate is selected from the group of well bottom, particle, bead, magnetic bead, porous member, non-porous member, solid member, microchannel, microchamber, reservoir, or combination thereof.

44.-48. (canceled)

49. A method of capturing a target substance, comprising:
providing the capture device of claim 41, wherein the R² is the capture entity; and
contacting a target substance to the capture moiety such that the target substance is captured.

50. The method of claim 49, wherein at least one of:
the capture entity is selected from the group of antibody, polypeptide, protein, aptamer, ligand, or receptor;
the substrate is selected from the group of well bottom, particle, bead, magnetic bead, porous member, non-porous member, solid member, microchannel, microchamber, reservoir, or combination thereof; or
the target substance is selected from a circulating cell, nucleic acid, peptide, protein, extracellular vesicle, exosome, or analyte.

51.-52. (canceled)

53. A method of releasing a captured target substance, comprising:
providing the capture device of claim 41, wherein the R² is the capture entity having a target substance associated therewith; and
irradiating the photocleavable heterobifunctional linker with light that cleaves the linker, thereby releasing the target substance from the substrate.

54. The method of claim 53, wherein the irradiating is with light having a wavelength greater than about 380 nm.

55. (canceled)
